SEQUENCE LISTING (37 sequences)

SEQ ID NO: 1 - DNA, 2403 bp, Homo sapiens
ctgcatttatttgccttgatccagcctgggagaagtcaggatagactttgggctgcttggccctggaggcagcttgagctgggactggggtggggggctcctgaggggctgcctaggacactgcagcttttgtgccttctccctgctgccaacacccccacacacactgctgcagccactctaaagccctttgtctttcattgcttagtcaccccctttgtcctcatctcaaataggggagtggaaaggggcagtagagttctctggtgatagctcctcttgcccctgccccttctggtctcccaccctttgtccgactcctctagtcccagccccgttggcttagaaccagggtcaggcaagtggtgggtcaagaggtgggtctggcagtcacaagggggtgggtgatccaggaagtgataggcaccagggcaggtattaccgacctgagcaggaagggagggggaaaggaagtattctgacggatatgatatgcgggggacaggaggtgacaaagcagagtgaataggggaatagaggcaagaggaggtggtccacttctgggaaaggaaagagactgctgactgcactctccttcctggggatttcctggggaaacaagcagccagaggatggggtgagcagaaattgcccctacttctgaacccttccttgccttgagagttcatacccaagacctcttttccgagttccctcctatccaaagccaaaggaataatttgcttcctttccctaacaccacctcttcctccccagccactttccccaccccaggcaatggatttctcccagtaccctaatttccctatatgcacaatgctgtctccaccctctccctgccccagggagaattaaaaagaaaagatgactagatattccaggaaccactgggttctcagagcaaggtggggtggatggtgggagccaggtggggattctcccagattgatactgggtgaatctgggttcctgagagcaagtcttgcctatgctgggggctggctgacttgaggctgggggagggtttagggcagttgggagtgggtaggagcagggccaaaagcctgggggaagctactgggagctgggccagggaaatggggagtcaggaagtggggagggggaaccctggggggaaatggaggcggaatggctgttctgggctttggagggggtgggtagtggtaactcaggaagggggatcctgagggagagaagggacgttagaaaagaggaggtgccaccctggatccgccttctataaaaggaaaagtcgttaacccctcctgccttgtcatctgccgcctctgttatgttcattccaagcaggatcatcctacctttgggcagtcaactccctgatcactgtctccttgcctcccccaatgttctgccttttttactcttcccagctgctcagttctatcctgagccatgtcaagctacctcttttatttgttcttccctcttgatgcctccttacctgttccctaccctcttttctcaggcagctcactcagtcccctcagccctggaaaccagccactagggccaaagggcagcatgagggagccttgagaaaagagaagccatggtaggttagactataagagcaggaattctcccaggaccgtgatcctatctgtgcatgccggccaggccctttccctcactctctgcctctcctggggctctgtcccaccaaaaagggaaagagacagctgagggctgattgtggggtttgggaaaaggctatgtcatcagctggcccagtgcctattatccattcggctgctagagattcccctcccctgggcaagtcccatttttttgggaagcgatgatacacccatctgagtcccaccgacagagctcagctgagtggcttagagatcagccaatcaatcgcagaggctcaccatgcttaaaagagctggcgcggagagaggctggggagaacccacagggagacccacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatg

SEQ ID NO: 2 - DNA, 2003 bp, Homo sapiens
gtgggtgatccaggaagtgataggcaccagggcaggtattaccgacctgagcaggaagggagggggaaaggaagtattctgacggatatgatatgcgggggacaggaggtgacaaagcagagtgaataggggaatagaggcaagaggaggtggtccacttctgggaaaggaaagagactgctgactgcactctccttcctggggatttcctggggaaacaagcagccagaggatggggtgagcagaaattgcccctacttctgaacccttccttgccttgagagttcatacccaagacctcttttccgagttccctcctatccaaagccaaaggaataatttgcttcctttccctaacaccacctcttcctccccagccactttccccaccccaggcaatggatttctcccagtaccctaatttccctatatgcacaatgctgtctccaccctctccctgccccagggagaattaaaaagaaaagatgactagatattccaggaaccactgggttctcagagcaaggtggggtggatggtgggagccaggtggggattctcccagattgatactgggtgaatctgggttcctgagagcaagtcttgcctatgctgggggctggctgacttgaggctgggggagggtttagggcagttgggagtgggtaggagcagggccaaaagcctgggggaagctactgggagctgggccagggaaatggggagtcaggaagtggggagggggaaccctggggggaaatggaggcggaatggctgttctgggctttggagggggtgggtagtggtaactcaggaagggggatcctgagggagagaagggacgttagaaaagaggaggtgccaccctggatccgccttctataaaaggaaaagtcgttaacccctcctgccttgtcatctgccgcctctgttatgttcattccaagcaggatcatcctacctttgggcagtcaactccctgatcactgtctccttgcctcccccaatgttctgccttttttactcttcccagctgctcagttctatcctgagccatgtcaagctacctcttttatttgttcttccctcttgatgcctccttacctgttccctaccctcttttctcaggcagctcactcagtcccctcagccctggaaaccagccactagggccaaagggcagcatgagggagccttgagaaaagagaagccatggtaggttagactataagagcaggaattctcccaggaccgtgatcctatctgtgcatgccggccaggccctttccctcactctctgcctctcctggggctctgtcccaccaaaaagggaaagagacagctgagggctgattgtggggtttgggaaaaggctatgtcatcagctggcccagtgcctattatccattcggctgctagagattcccctcccctgggcaagtcccatttttttgggaagcgatgatacacccatctgagtcccaccgacagagctcagctgagtggcttagagatcagccaatcaatcgcagaggctcaccatgcttaaaagagctggcgcggagagaggctggggagaacccacagggagacccacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatg

SEQ ID NO: 3 - DNA, 1603 bp, Homo sapiens
ggatttctcccagtaccctaatttccctatatgcacaatgctgtctccaccctctccctgccccagggagaattaaaaagaaaagatgactagatattccaggaaccactgggttctcagagcaaggtggggtggatggtgggagccaggtggggattctcccagattgatactgggtgaatctgggttcctgagagcaagtcttgcctatgctgggggctggctgacttgaggctgggggagggtttagggcagttgggagtgggtaggagcagggccaaaagcctgggggaagctactgggagctgggccagggaaatggggagtcaggaagtggggagggggaaccctggggggaaatggaggcggaatggctgttctgggctttggagggggtgggtagtggtaactcaggaagggggatcctgagggagagaagggacgttagaaaagaggaggtgccaccctggatccgccttctataaaaggaaaagtcgttaacccctcctgccttgtcatctgccgcctctgttatgttcattccaagcaggatcatcctacctttgggcagtcaactccctgatcactgtctccttgcctcccccaatgttctgccttttttactcttcccagctgctcagttctatcctgagccatgtcaagctacctcttttatttgttcttccctcttgatgcctccttacctgttccctaccctcttttctcaggcagctcactcagtcccctcagccctggaaaccagccactagggccaaagggcagcatgagggagccttgagaaaagagaagccatggtaggttagactataagagcaggaattctcccaggaccgtgatcctatctgtgcatgccggccaggccctttccctcactctctgcctctcctggggctctgtcccaccaaaaagggaaagagacagctgagggctgattgtggggtttgggaaaaggctatgtcatcagctggcccagtgcctattatccattcggctgctagagattcccctcccctgggcaagtcccatttttttgggaagcgatgatacacccatctgagtcccaccgacagagctcagctgagtggcttagagatcagccaatcaatcgcagaggctcaccatgcttaaaagagctggcgcggagagaggctggggagaacccacagggagacccacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatg

SEQ ID NO: 4 - DNA, 1203 bp, Homo sapiens
tagtggtaactcaggaagggggatcctgagggagagaagggacgttagaaaagaggaggtgccaccctggatccgccttctataaaaggaaaagtcgttaacccctcctgccttgtcatctgccgcctctgttatgttcattccaagcaggatcatcctacctttgggcagtcaactccctgatcactgtctccttgcctcccccaatgttctgccttttttactcttcccagctgctcagttctatcctgagccatgtcaagctacctcttttatttgttcttccctcttgatgcctccttacctgttccctaccctcttttctcaggcagctcactcagtcccctcagccctggaaaccagccactagggccaaagggcagcatgagggagccttgagaaaagagaagccatggtaggttagactataagagcaggaattctcccaggaccgtgatcctatctgtgcatgccggccaggccctttccctcactctctgcctctcctggggctctgtcccaccaaaaagggaaagagacagctgagggctgattgtggggtttgggaaaaggctatgtcatcagctggcccagtgcctattatccattcggctgctagagattcccctcccctgggcaagtcccatttttttgggaagcgatgatacacccatctgagtcccaccgacagagctcagctgagtggcttagagatcagccaatcaatcgcagaggctcaccatgcttaaaagagctggcgcggagagaggctggggagaacccacagggagacccacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatg

SEQ ID NO: 5 - DNA, 803 bp, Homo sapiens
aaaagagaagccatggtaggttagactataagagcaggaattctcccaggaccgtgatcctatctgtgcatgccggccaggccctttccctcactctctgcctctcctggggctctgtcccaccaaaaagggaaagagacagctgagggctgattgtggggtttgggaaaaggctatgtcatcagctggcccagtgcctattatccattcggctgctagagattcccctcccctgggcaagtcccatttttttgggaagcgatgatacacccatctgagtcccaccgacagagctcagctgagtggcttagagatcagccaatcaatcgcagaggctcaccatgcttaaaagagctggcgcggagagaggctggggagaacccacagggagacccacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatg

SEQ ID NO: 6 - DNA, 403 bp, Homo sapiens
cacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatg

SEQ ID NO: 7 - DNA, 21 bp, Homo sapiens
tttccctggcaaggactatga

SEQ ID NO: 8 - DNA, 17 bp, Homo sapiens
aatggcgtgagtcgggc

SEQ ID NO: 9 - DNA, 26 bp, Homo sapiens
tgatctcttttggaattaaggagcat

SEQ ID NO: 10 - DNA, 23 bp, Homo sapiens
atgggcatctcctccataatttg

SEQ ID NO: 11 - DNA, 19 bp, Homo sapiens
gcaaaccttcaaggcagcc

SEQ ID NO: 12 - DNA, 19 bp, Homo sapiens
tgctgtttgcctcggacat

SEQ ID NO: 13 - DNA, 33 bp, Homo sapiens
gcgcgctcgagctgcatttatttgccttgatcc

SEQ ID NO: 14 - DNA, 33 bp, Homo sapiens
gcgcgaagcttggcactggtactgcagctggga

SEQ ID NO: 15 - DNA, 33 bp, Homo sapiens
gcgcgctcgaggtgggtgatccaggaagtgata

SEQ ID NO: 16 - DNA, 36 bp, Homo sapiens
gcgcgctcgaggatttctcccagtaccctaatttcc

SEQ ID NO: 17 - DNA, 33 bp, Homo sapiens
gcgcgctcgagtagtggtaactcaggaaggggg

SEQ ID NO: 18 - DNA, 33 bp, Homo sapiens
gcgcgctcgagaaaagagaagccatggtaggtt

SEQ ID NO: 19 - DNA, 33 bp, Homo sapiens
gcgcgctcgagcacatatgcacgagagagacag

SEQ ID NO: 20 - DNA, 22 bp, Homo sapiens
ccttcctggggatttcctgggg

SEQ ID NO: 21 - DNA, 22 bp, Homo sapiens
ccccaggaaatccccaggaagg

SEQ ID NO: 22 - DNA, 22 bp, Homo sapiens
ccttcctggagatttcctgggg

SEQ ID NO: 23 - DNA, 22 bp, Homo sapiens
ccccaggaaatctccaggaagg

SEQ ID NO: 24 - DNA, 20 bp, Homo sapiens
cattgcttagtcaccccctt

SEQ ID NO: 25 - DNA, 20 bp, Homo sapiens
aagggggtgactaagcaatg

SEQ ID NO: 26 - DNA, 20 bp, Homo sapiens
cattgcttgggcaccccctt

SEQ ID NO: 27 - DNA, 20 bp, Homo sapiens
aagggggtgcccaagcaatg

SEQ ID NO: 28 - DNA, 27 bp, Homo sapiens
ggtccacttctgggaaaggaaagagac

SEQ ID NO: 29 - DNA, 27 bp, Homo sapiens
gtctctttcctttcccagaagtggacc

SEQ ID NO: 30 - DNA, 27 bp, Homo sapiens
ggtccacatatgggaaaggaaagagac

SEQ ID NO: 31 - DNA, 27 bp, Homo sapiens
gtctctttcctttcccatatgtggacc

SEQ ID NO: 32 - DNA, 37 bp, Homo sapiens
ctttgtctttcattgcttgggcaccccctttgtcctc

SEQ ID NO: 33 - DNA, 37 bp, Homo sapiens
gaggacaaagggggtgcccaagcaatgaaagacaaag

SEQ ID NO: 34 - DNA, 38 bp, Homo sapiens
caagaggaggtggtccacatatgggaaaggaaagagac

SEQ ID NO: 35 - DNA, 38 bp, Homo sapiens
gtctctttcctttcccatatgtggaccacctcctcttg

SEQ ID NO: 36 - DNA, 32 bp, Homo sapiens
cactctccttcctggagatttcctggggaaac

SEQ ID NO: 37 - DNA, 32 bp, Homo sapiens
gtttccccaggaaatctccaggaaggagagtg

(19) United States
(12) Patent Application Publication
(10) Pub. No.: US 2010/0196957 A1
(43) Pub. Date: Aug. 5, 2010

(54) CLONING AND CHARACTERIZATION OF 5' FLANKING REGIONS OF A HUMAN AGGRECANASE-1 GENE

(76) Inventor: Chandrika Saidapet Kumar, Acton, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/577,345

(22) Filed: Oct. 12, 2009

Related U.S. Application Data
(63) Continuation of application No. 10/578,469, filed on May 22, 2007, now abandoned.
(60) Provisional application No. 60/517,829, filed on Nov. 6, 2003.

(51) Int. Cl. C12P 21/06; C07H 21/04; C12N 15/63; C12N 5/02; C12P 19/34

(52) U.S. Cl. 435/69.1; 536/23.1; 435/320.1; 435/325; 435/91.1

(57) ABSTRACT

The present invention relates to an isolated polynucleotide molecule comprising an Agg-1 promoter gene and methods of using the same. The invention also provides a method for producing an expression product encoded by a nucleic acid through use of an Agg-1 promoter.

### BACKGROUND OF THE INVENTION

Cartilage is composed of 65-80% water, collagen, proteoglycans and chondrocytes. Aggrecan and type II collagen are the most abundant of proteoglycans found in articular cartilage. Aggrecan provides compressive resistance whereas type II collagen network provides resistance to shear and tensile force. The degradation of Aggrecan and type II collagen are the key features of arthritis. Specifically, the loss of Aggrecan from articular cartilage is one of the earliest pathophysiological hall marks of OA.

Aggrecan degrading metallo proteases involved in OA are disclosed in U.S. Pat. No. 6,451,575. Aggrecanase-1 (Agg-1) and Aggrecanase-2 (Agg-2) are members of the ADAMTS (A Disintegrin And Metalloproteinase with Thrombospondin motifs) family. Both Agg-1 (ADAMTS4) and Agg-2 (ADAMTS11) and a third member, ADAM-TSI cleave the Glu¹⁸⁷¹-Leu¹⁸⁷² bond within the chondroitin sulfate attachment domain of Aggrecan and are expressed in cartilaginous tissues. See Kahn et al., J Clin Invest pp. 1335-1337 (2000); Konttinen et al., Ann Rheum Dis, Vol. 58, No. 10, pp. 691-697 (1999); Hurskainen et al., J Biol Chem, Vol. 274, No. 36, pp. 25555-25563 (1999); Abbaszade, Liu and Yang, J Biol Chem, Vol. 274, No. 33, pp. 23443-23450 (1999); Tortorella et al., J Biol Chem, Vol. 275, No. 24, pp. 18566-18573 (2000); Kuno et al, FEBS Lett, Vol. 478, pp. 241-245 (2000); Rees et al., Biochem J, Vol. 350, Pt. 1, pp. 181-188 (2000); and Matthews et al., J Biol Chem, Vol. 275, No. 30, pp. 22695-22703 (2000). The cleavage products accumulate in the synovial fluid of joints. Type II collagen degrading enzymes have been identified among the metalloprotease (MMP) family and articular chondrocytes produce collagenase-1, 2 and 3. See Shlopov et al., Arthritis Rheum, Vol. 40, No. 11, pp. 2065-2074 (1997). Although collagen fibrils are substrates for all three collagenases, collagenase-3 (MMP-13) is more efficient than other collagenases in degrading type II collagen. See Reboul et al., J Clin Invest, Vol. 97, No. 9, pp. 2011-2019 (1996). An Agg-1 promoter region is shown in and JP2001245663A.

The pathological conditions of OA can be modeled in vitro by stimulation of primary human articular chondrocytes (HAC), chondrocyte cell lines (C28A2 or SW1353) or non-chondrocytic cell lines (HeLa, NIH 3T3 or 293) with growth factors, such as PDGF or cytokines, such as Interleukin 1β (IL-1β) or Oncostatin M (OSM) or a combination of and OSM. See Billington, Clark and Cawston, Biochem J, Vol 336, Pt. 1, pp. 207-212 (1998); and Cawston et al., Biochem Biophys Res Commun, Vol. 215, No. 1, pp. 377-385 (1995). IL-1β alone potently induces Agg-1, MMP-13, the two key cartilage degrading enzymes and alsoiNOS, which is a mediator of cytotoxicity and inflammation in human articular cartilage. See Taylor et al., J Biol Chem, Vol. 273, No. 24, pp. 15148-15156 (1998); Vincenti and Brinckerhoff, Arthritis Res, Vol. 4, No. 3, pp. 157-164 (2002); Bau at al., Arthritis Rheum, Vol. 46, No. 10, pp. 2648-2657 (2002); Flannery et al., Matrix Biol, Vol. 18, No. 3, pp. 225-237 (1999); and Tardif et al., Arthritis Rheum, Vol. 42, No. 7, pp. 1147-1158 (1999). In order to understand how these genes regulate cartilage degradation, it is important to understand how their transcriptional activity is regulated by cytokines and growth factors. This necessitates the characterization of promoter regions of these key genes. The promoter regions for INOS and MMP-13 have been cloned and characterized, by several groups [see Genstar et al., Proc Natl Aced Sci USA, Vol 98, pp. 8638-8643 (2001); Yu, Zhang and Kone, Biochem J, Vol. 367, Pt. 1, pp. 97-105 (2002); Tardif et al., Bioichem J, Vol. 323, Pt. 1, pp. 13-16 (1997); Pendas et al, Genomics, Vol. 40, No. 2, pp. 222-233 (1997); and Li, Dehnade and Zafarullah, J Immunol, Vol. 166, No. 5, pp. 3491-3498 (2001)] however there is very limited information available for the Agg-1 promoter.

Thus, there is a need for characterization and control of the active mechanisms involved in degenerative joint diseases, such as OA, particularly of nucleic acids and compounds. There is also a need for useful tools for diagnosing diseases or for monitoring the efficacy of therapeutic agents that have been developed to treat inflammation and joint diseases.

### SUMMARY OF THE INVENTION

This invention relates to a substantially purified nucleic acid molecule comprising an Agg-1 promoter gene.

This invention also relates to a nucleic acid molecule having a sequence selected from the group consisting of:
(a) a nucleic acid sequence substantially homologous to that of SEQ ID NOs: 1-6, or a fragment thereof;
(b) a nucleic acid sequence substantially complementary to the nucleic add sequence of (a) or a fragment thereof;
(c) a nucleic add sequence that hybridizes to the nucleic acid sequences of (a) or (b) or fragments thereof, and
(d) a nucleic acid sequence identical to any of the sequences of (a), (b) or (c), with the proviso that any T may have been replaced by U or I.

The invention also relates to expression vectors comprising the aforementioned nucleic acid sequences and host cells transformed with these expression vectors.

The invention also relates to methods for detecting test agents which modulate activity of Aggrecanase (Agg): promoters comprising contacting a host cell transformed with an expression vector comprising an Agg promoter operably-linked to a reporter gene with the test agent and comparing the level of reporter expressed in the presence of the test agent to the level of reporter expressed in its absence.

One aspect of the invention provides methods of screening for test compounds that regulate transcription of an Agg-1 promoter gene by:
(a) contacting a host cell in which the Agg-1 promoter gene disclosed herein is operably-linked to a reporter gene with a test medium containing the test compound under conditions which allow for expression of the reporter gene;
(b) measuring the expression of the reporter gene in the presence of the test medium;
(c) contacting the host cell with a control medium which does not contain the test compound but is otherwise identical to the test medium in (a), under conditions identical to those used in (a);
(d) measuring the expression of reporter gene in the presence of the control medium; and
(e) relating the difference in expression between (b) and (d) to the ability of the test compound to regulate transcription of an Agg-1 promoter gene.

Another aspect of this invention provides methods of measuring the ability of a test compound to modulate Agg-1 promoter transcription by
(a) contacting a host cell in which an Agg-1 promoter gene disclosed herein is operably-linked to a reporter gene with an inducer of Agg-1 promoter activity under conditions which allow for expression of the reporter gene;
(b) measuring the expression of the reporter gene in the absence of the test compound;
(c) exposing the host cells to the test compound either prior to, simultaneous with, or after contacting, the host cells with the inducer;
(d) measuring the expression of the reporter gene in the presence of the test compound; and
(e) relating the difference in expression between (b) and (d) to the ability of the test compound to modulate the transcription of Agg-1 promoters.

The invention also relates to methods of modulating articular cartilage degeneration in a mammal comprising introducing into chondrocytes or synoviocytes a vector comprising a nucleic acid sequence encoding an Agg promoter operably-linked to a nucleic acid sequence encoding a protein, polypeptide, hormone, ribozyme or antisense RNA, which decreases cartilage degradation and/or inhibits proteolytic enzyme activity or expression.

The invention also relates to the use of a vector comprising a nucleic acid sequence encoding an Agg promoter operably-linked to a nucleic acid sequence encoding a protein, polypeptide, hormone, ribozyme or antisense RNA, which decreases cartilage degradation and/or inhibits proteolytic enzyme activity or expression, in the preparation of a medicament for modulating articular cartilage degeneration in a mammal.

In one aspect, the invention provides a method of screening for therapeutic compounds which modulate articular cartilage degeneration comprising:
(a) providing a tissue sample from a subject known to have articular cartilage degeneration, wherein at least one cell in said tissue sample is capable of producing one or more nucleic acid sequences selected from the group consisting of SEQ ID NOs. 1-6, having promoter activity;
(b) contacting said tissue sample with said candidate therapeutic agent;
(c) measuring the activity of said nucleic acid sequences in the tissue sample;
(d) comparing the activity of said nucleic acid sequences to the activity of said nucleic acid sequences in a tissue sample from a control subject; and
(e) identifying a difference in activity levels of the Agg-1 promoter gene, if present, in the diseased tissue and control tissue, thereby identifying a therapeutic agent for treating articular cartilage degeneration.

In another aspect, the invention encompasses a method of treating, preventing or delaying onset of disorders associated with inflammation comprising administering to a patient suffering from or at risk for developing disorders associated with inflammation an agent that inhibits or decreases the induction or activity of one or more nucleic add sequences selected from the group consisting of SEQ ID NOs: 1-6.

In another aspect, the invention relates to a use of an agent that inhibits or decreases the induction or activity of one or more nucleic acid sequences selected from the group consisting of SEQ ID NOs: 1-6, in the preparation of a medicament for treating, preventing or delaying onset of disorders associated with inflammation in a patient suffering from or at risk for developing disorders associated with inflammation.

The invention also relates to deletion variants of an Agg-1 promoter gene. Preferred deletion variants include SEQ ID NOs: 2-6, most preferably SEQ ID NOs: 2-5 and complements thereof.

The invention also relates to antibodies which are immunospecific for one or more of Agg-1 promoter genes or constructs described herein.

The invention also relates to transgenic or chimeric animals whose cells express a heterologous gene under the transcriptional control of an Agg-1 promoter gene of the present invention.

### BRIEF DESCRIPTION OF THE DRAWINGS

FIG. 1 shows the 5′ flanking region of human Agg-1 promoter genes. Sequences for the transcription factors NFkB, AP-1, STAT3, NF1 and c-krox are shown underlined. The CAAT and TATAA boxes are shown underlined.

### DETAILED DESCRIPTION OF THE INVENTION

All patent applications, patents and literature references cited herein are hereby incorporated by reference in their entirety.

The invention relates to novel promoters for Agg enzymes, nucleic acid constructs comprising such promoters Operatively-linked to genes encoding a gene product, such as a protein, polypeptide, hormone, ribozyme or antisense RNA, recombinant cells comprising such nucleic add constructs, methods of screening for therapeutic drugs using such cells, and Agg-specific gene expression using the novel promoter sequences of the invention.

Before describing the invention in greater detail the following definitions are set forth to illustrate and define the meaning and scope of the terms used to describe the invention herein.

The term “nucleic acid molecule” is meant to include DNA, RNA and mixed DNA-RNA sequences. In addition to the typically found A, T, U, (and C residues, including related residues, such as, e.g., inosine (I).

An “isolated” nucleic acid molecule or isolated enzymes are nucleic acid molecules or enzymes that, by the hand of man, exists apart from its native environment and is therefore not a product of nature. An isolated nucleic acid molecule or enzyme may exist in a purified form or in a non-native environment such as, for example, in a recombinant cell.

The term “polynucleotide” or “gene”, as used herein, refer to a polymeric form of nucleotides of any length, either ribonucleotides or deoxyribonucleotides. Thus, this term includes double- and single-stranded DNA, triplex DNA, as well as double- and single-stranded RNA. It also includes modified, e.g., by methylation and/or by capping, and unmodified forms of the polynucleotide.

The term “recombinant polynucleotide”, as used herein, refers to a polynucleotide of genomic, cDNA, semisynthetic or synthetic origin which, by virtue of its origin or manipulation: (1) is not associated with all or a portion of the polynucleotide with which it is associated in nature; and/or (2) is linked to a polynucleotide other than that to which it is linked in nature.

“Substantial homology” in the nucleic acid context means that the segments, or their complementary strands, when compared, are the same when properly aligned, with the appropriate nucleotide insertions and deletions, in at least about 60% of the nucleotides, typically, at least about 70%, more typically, at least about 80%, usually; at least about 90%, and more usually, at least, about 95-98% of the nucleotides. Alternatively, substantial homology exists between two segments when the segments or their complementary strands will hybridize under stringent hybridization conditions to a template strand. Selective hybridization exists when the hybridization is more selective than total lack of specificity. See, Kanehisa, Nucleic Acids Res, Vol. 12, No. 1, Pt. 1, pp. 203-213 (1984).

The term “regulatory sequences” refers to sequences which influence the specificity and/or level of expression, e.g., in the sense that they confer cell and/or tissue specificity. Such regions can be located upstream of the transcription initiation site, but can also be located downstream of it, e.g., in transcribed but not translated leader sequences.

The term “promoter” refers to a nucleic add sequences that function to control the transcription of one or more genes, located upstream with respect to the direction of transcription initiation site of the gene, and is structurally identified by the presence of a binding site for DNA-dependent RNA polymerase, transcription initiation sites and any other DNA sequences including, but not limited to, transcription factor binding sites, repressor and activator protein binding sites and other sequences of nucleotides known to one of skill in the art to act directly or indirectly to regulate the amount of transcription from the promoter.

The term “promoter activity” refers to the extent of transcription or induction of a gene that is operably-linked to the promoter whose promoter activity is being measured. The promoter activity may be measured directly by measuring the amount of RNA transcript produced, e.g., by Northern blot or indirectly by measuring the product coded for by the RNA transcript, such as when a reporter gene is linked to the promoter.

“Induction” refers to an increase in the expression of a gene due to the activity of a regulatory protein.

“Transcription” refers to synthesis of an RNA copy from a sequence of DNA (a gene); the first step in gene expression.

The term “substantially purified” refers to a nucleic acid sequence, polypeptide, protein or other compound which is essentially free, i.e., is more than about 50% free of, preferably more than about 60% free of, preferably 75% free and more preferably about 90% free of, the polynucleotides, proteins, polypeptides and other molecules that the nucleic acid, polypeptide, protein or other compound is naturally associated with.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue, i.e., GAU and GAC triplets each encode Asp.

A “variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention. As a result of alterations, a variant protein has an amino acid sequence which is at least 95% identical to a protein encoded by an Agg-1 promoter gene, preferably, at least 97% identical, more, preferably at least 98% identical, most preferably at least 99% identical. Variant sequences which are at least 95% identical have no more than 5 alterations, i.e., any combination of deletions, insertions or substitutions, per 100 amino acids encoded by an Agg-1 promoter gene. Sequences are aligned for calculation of percent identity using the method of the software basic local alignment search tool in the BLAST network service (the National Center for Biotechnology Information, Bethesda, Md.) which employs the method of Altschul et al., J Mol Biol, Vol. 215, No. 3, pp. 403-410 (1990). Identities are calculated by the Align program (DNAstar, Inc.). In all cases, internal gaps and amino add insertions in the candidate sequence as aligned are not ignored when making the identity calculation.

The term “operably-linked” refers to linkage of a nucleic acid segment to another nucleic acid segment in such a way as to allow the segments to function in their intended manners. A nucleic acid sequence encoding a gene product is operably-linked to a regulatory sequence when it is ligated to the regulatory sequence, such as, e.g., promoters, enhancers and silencers, in a manner which allows modulation of transcription of the nucleic acid sequence, directly or indirectly. For example, a nucleic acid sequence is operably-linked to a promoter when it is ligated to the promoter downstream with respect to the transcription initiation site of the promoter, in the correct reading frame with respect to the transcription initiation site and allows transcription elongation to proceed through the nucleic acid sequence. An enhancer or silencer is operably linked to a nucleic acid sequence coding for a gene product when it is ligated to the nucleic acid sequence in such a manner as to increase or decrease respectively the transcription of the nucleic acid sequence. Enhancers and silencers may be located upstream, downstream or embedded within the coding regions of the nucleic acid sequence. Linkage of nucleic acid sequences to regulatory sequences is typically accomplished by ligation at suitable restriction sites or adapters or linkers inserted in lieu thereof using restriction endonucleases known to one of skill in the art.

The terms “transcriptional regulator” or “transcription factor” are used interchangeably herein and refer to a biochemical element that acts to prevent or inhibit the transcription of a promoter-driven DNA sequence under certain environmental conditions. e.g., a repressor or nuclear inhibitory protein, or to permit or stimulate the transcription of the promoter-driven DNA sequence under certain environmental conditions, e.g., an inducer or an enhancer.

The term “induction” refers to an increase in gene transcription or expression brought about by a transcriptional inducer, relative to some basal level of transcription.

The term “repression” refers to a decrease in gene transcription or expression brought about by a transcriptional repressor, relative to some basal level of transcription.

The term “basal promoter activity” or “basal level” are used interchangeably herein and refer to activity of reporter in the absence of a transcriptional regulator.

The term “heterologous” refers to nucleic add molecules do not occur naturally as part of the genomic DNA or RNA sequence in which it is present, of that is found in a cell or locations in the genome or nucleic acid sequences that differ from where they are found in nature. Heterologous nucleic add molecules are not endogenous to the cell into which it is introduced, but has been obtained from another cell or synthetically or recombinantly produced. Generally, though not necessarily, such molecules encode RNA and proteins that are not normally produced by the cell in which the sequence is transcribed or expressed. Any nucleic acid molecule that one of skill in the art would recognize as heterologous or foreign to the cell in which it is expressed is herein encompassed by the term heterologous. Examples of heterologous nucleic acid molecules include, but are not limited to, DNA that encodes proteins, polypeptides, receptors, reporter genes, transcriptional and translational regulatory sequences, selectable or traceable marker proteins, such as a protein that confers drug resistance, RNA including mRNA and antisense RNA and ribozymes.

A “reporter gene” is a nucleic add molecule that expresses a detectable gene product, which may be RNA or protein. The detection may be accomplished by any method known to one of skill in the art. For example, detection of mRNA expression may be accomplished by using. Northern blots and detection of protein may be accomplished by staining with antibodies specific to the protein. Preferred reporter genes are those that are readily detectable. A reporter gene may be operably linked in a DNA construct with a regulatory nucleic acid sequence such that detection of the reporter gene product provides a measure of the transcriptional activity of the regulatory sequence. Examples of reporter genes include, but are not limited to, those coding for chloramphenicol acetyl transferase (CAT), luciferase, beta.-galactosidase and alkaline phosphatase.

An “expression vector” is any genetic element, e.g., a plasmid, a chromosome, a virus, behaving either as an autonomous unit of polynucleotide replication within a cell, i.e., capable of replication under its own control, or being rendered capable of replication by insertion into a host all chromosome, having attached to it another polynucleotide segment, so as to bring about the replication and/or expression of the attached segment. Suitable vectors include, but are not limited to, plasmids, bacteriophages and cosmids. Vectors may contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors may be capable of directly expressing gene products encoded therein without ligation or integration of the vector into host cell DNA sequences.

The terms “transformed” or “transfected” are used interchangeably herein and refer to the process by which exogenous nucleic acid sequences are transferred or introduced into an appropriate host cell. Such transfected cells include stably transfected cells wherein the inserted DNA is rendered capable of replication in the host cell. Typically, stable transfection requires that the exogenous nucleic acid sequence be transferred along with a selectable marker gene, such as, e.g., a gene that confers antibiotic resistance, which enables the selection of the stable transfectants. This marker gene may be ligated to the exogenous sequence or be provided independently by simultaneous cotransfection along with the exogenous sequence. Transfected cells also include transiently expressing cells that are capable of expressing the nucleic acid sequence for limited periods of time. The host cell may be a prokaryotic or eukaryotic cell. The transfection procedure depends on the host cell being transfected. It can include packaging the polynucleotide in a virus as well as direct uptake of the polynucleotide. Transformation can result in incorporation of the inserted DNA into the genome of the host cell or the maintenance of the inserted. DNA within the host cell in plasmid form. Methods of transformation/transfection are well-known in the art and include, but are not limited to, direct injection, such as microinjection; viral infection, particularly replication-deficient adenovirus infection; electroporation; lipofection; calcium phosphate-mediated direct uptake; and the like. General methods, vectors, gene transfer and expression may be found in Kriegler, Gene Transfer and Expression: A Laboratory Manual, Stockton Press (1990).

The term “host cell” generally refers to prokaryotic or eukaryotic organisms and includes any transformable organism which is capable of expressing a protein and can be, or has been, used as a recipient for expression vectors or other transfer nucleic acid sequence. Chondrocyte cell lines, such as primary human articular chondrocyte cells, as well as nonchondrocytic cell lines, such as HeLa, NIH 3T3 or 293 are suitable host cells for expression vectors.

It is to be understood that this invention is intended to include other forms of expression vectors, host cells and transformation techniques which serve equivalent functions and which become known to the art hereto.

As noted above, the present invention relates to recombinant or heterologous polynucleotides comprising an Agg promoter gene. This invention provides polynucleotides having a sequence selected from the group consisting of:
(a) the nucleic acid sequence substantially homologous to that of SEQ ID NOs: 1-6 or a fragment thereof;
(b) a nucleic acid sequence substantially complementary to said nucleic acid sequence of (a), or a fragment thereof; and
(c) a nucleic acid sequence that hybridizes to said nucleic acid sequences of (a) or (b) or fragments thereof.

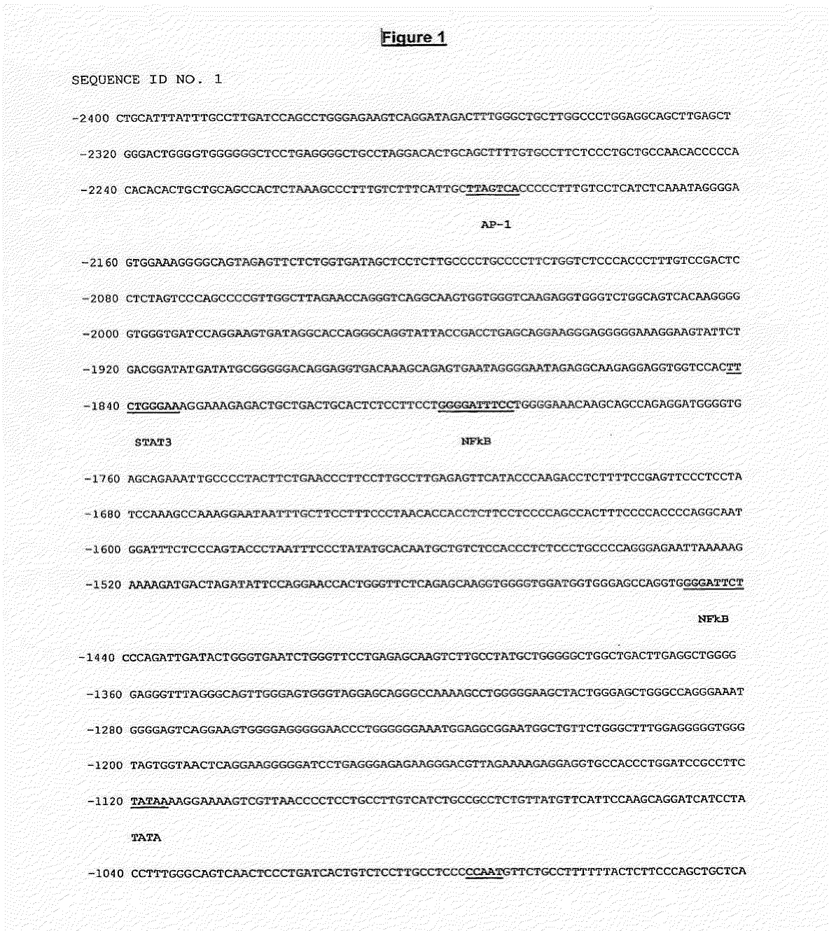

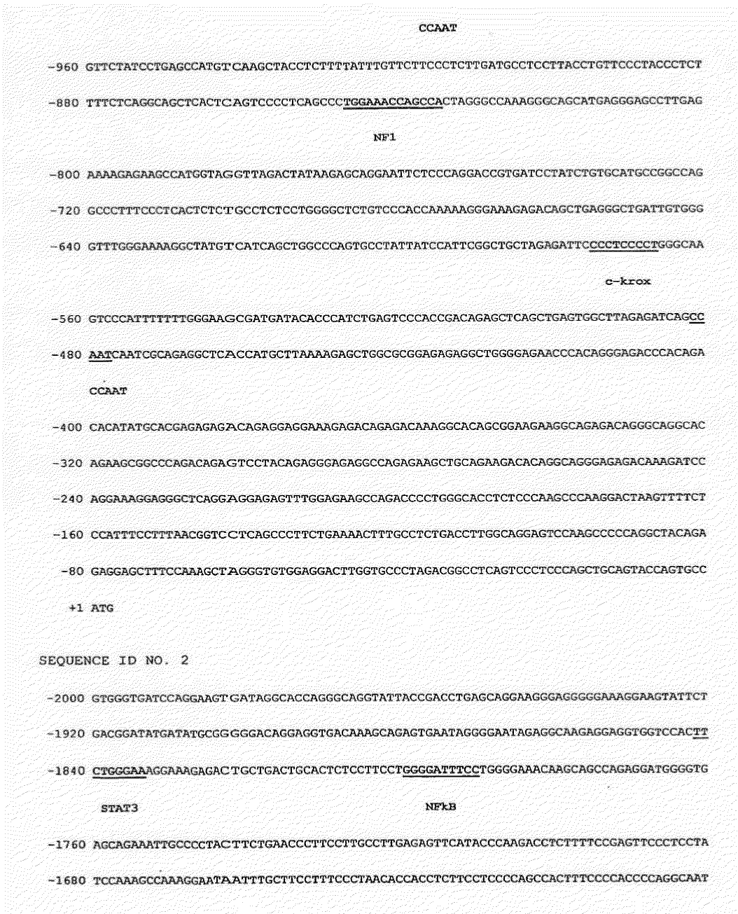

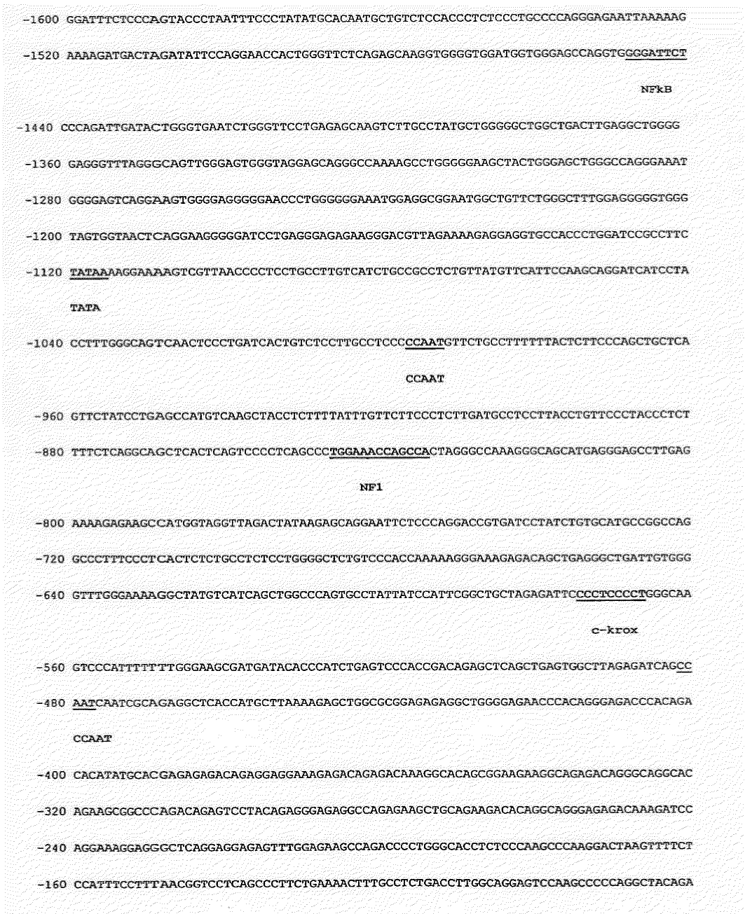

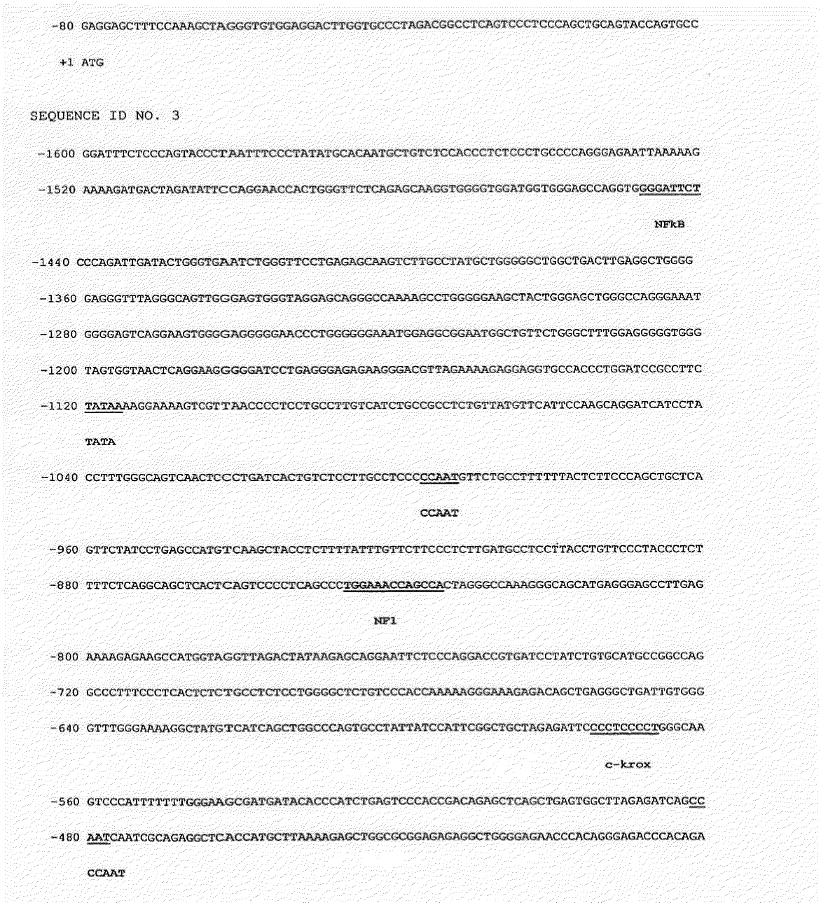

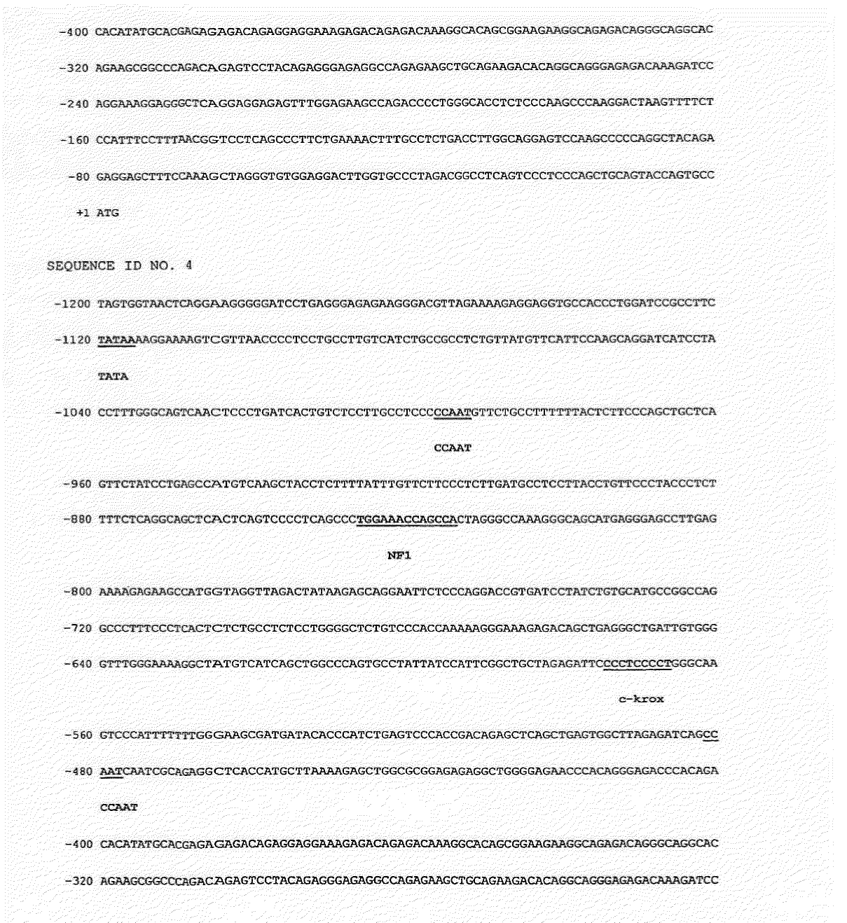

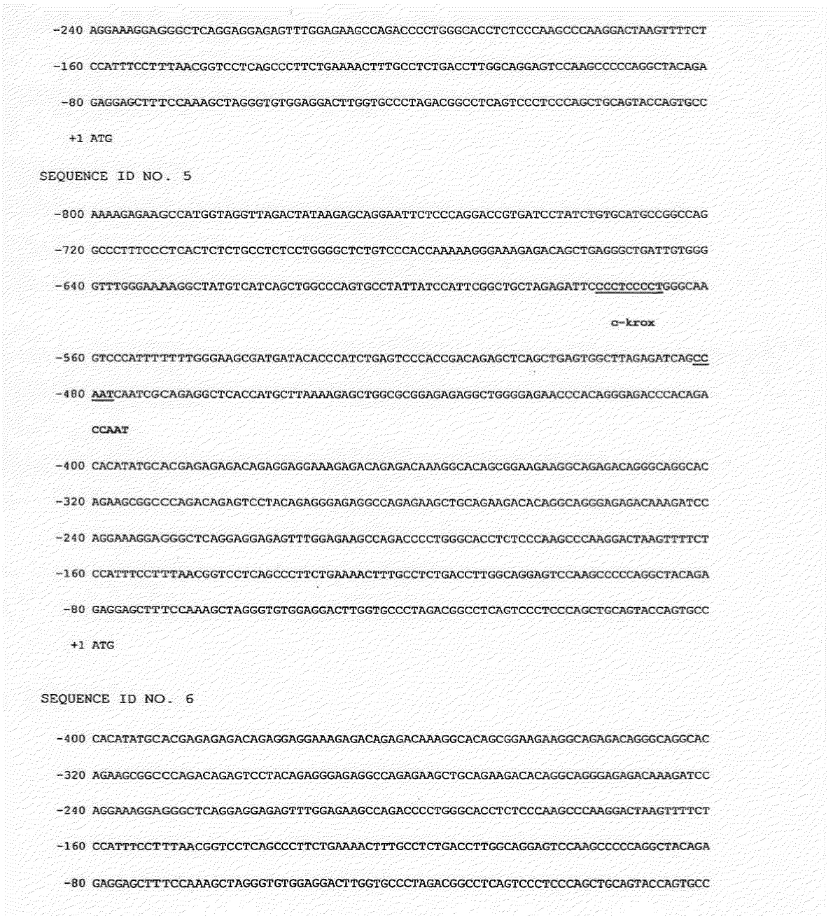

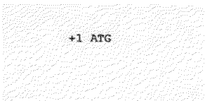

Preferably, such nucleic acid molecules will be substantially homologous to the nucleic acid sequences shown in FIG. 1, and more preferably to the sequence of SEQ ID NOs: 2-5.

The polynucleotides of the present invention, regardless of the length of the sequence itself, may be combined with other nucleic acid sequences on either the 5′ or 3′ ends of the sequences disclosed herein including, but not limited to, other promoters, enhancers, polyadenylation signals, restriction enzyme sites; multiple cloning sites, coding segments and the like, to create one or more nucleic add construct(s). Such polynucleotides may comprise a nucleic acid segment that contains multiple nucleic acid regions, including a nucleic acid sequence which is used to generate a gene product; such as an RNA or protein, restriction sites, etc. The overall length may vary considerably between such polynucleotides. Thus, a nucleic acid segment of almost any length may be employed, with the total length preferably being limited by the ease of preparation or use in the intended recombinant nucleic add protocol.

The polynucleotides of the present invention also encompass sequences that are capable of hybridizing to the nucleotide sequences of SEQ ID Nos: 1-6 under stringent conditions, preferably highly-stringent conditions. Hybridization conditions are based on the melting temperature, Tm, of a nucleic add binding complex or probe, as described in Kimmel and Berger, Methods Enzymol, Vol. 152, pp. 307-316 (1987). The term “stringent conditions”, as used herein, is the “stringency” which occurs within a range from about Tm−5 (5° C. below the melting temperature of the probe) to about 20° C. below Tm. As used herein, “highly-stiingent” conditions employ at least 0.2×SSC buffer and at least 65° C. As recognized in the art, stringency conditions can be attained by varying a number of factors, such as the length and nature, i.e., DNA or RNA, of the probe; the length and nature of the target sequence, the concentration of the salts and other components, such as formamide, dextran sulfate and polyethylene glycol, of the hybridization solution. All of these factors may be varied to generate conditions of stringency which are equivalent to the conditions listed above.

The present polynucleotides also encompass altered polynucleotides which encode an Agg-1 promoter gene and variants thereof. Such alterations include deletions, additions or substitutions. Such alterations may produce a silent change and result in an Agg-1 promoter gene having the same activity or binding properties as an Agg-1 promoter encoded by the unaltered polynucleotide. Such alterations may produce a nucleotide sequence possessing non-naturally occurring codons. Such alterations may also introduce new restriction or binding sites into the sequence or result in the production of an Agg-1 promoter variant. Typically, such alterations are accomplished using site-directed mutagenesis.

Also encompassed by the present invention, are single-stranded polynucleotides, hereinafter referred to as antisense polynucleotides, having sequences which have substantial homology or are complementary to an Agg-1 promoter gene of the invention. The term complementary as used herein refers to the natural binding of the polynucleotides under permissive salt and temperature conditions by base pairing.

The present invention also encompasses oligonucleotides that are used as primers in polymerase chain reaction (PCR) techniques to amplify transcripts of an Agg-1 promoter gene or fragments of such transcripts. Preferably, the primers comprise 18-30 nucleotides, more preferably 19-25 nucleotides. Preferably, the primers have a G+C content of 40% or greater. Such oligonucleotides are at least 95-98% complementary with a portion of the DNA strand, i.e., the sense strand, which encodes an Agg-1 promoter gene or fragment or its corresponding antisense strand. Preferably, the primer has at least 99% complementarity, more preferably 100% complementarity, with such sense strand or its corresponding antisense strand. Primers which have 100% complementarity with the antisense strand of a double-stranded Agg-1 promoter gene have a sequence which is identical to a sequence contained within the sense strand.

The present invention also encompasses oligonucleotides that are useful as hybridization probes for isolating and identifying cDNA clones and genomic clones of an Agg-1 promoter gene, fragments or allelic forms thereof. Such hybridization probes are also useful for detecting transcripts for mapping of an Agg-1 promoter gene. Preferably, such probes comprise at least 5 nucleotides, more preferably at least 10, most preferably from about 10-30 nucleotides. Such hybridization probes have a sequence which is at least 90% complementary with a sequence contained within the sense Strand of an Agg-1 promoter gene or with a sequence contained within its corresponding antisense strand. Such hybridization probes bind to the sense strand under stringent conditions. The identity of probes which are 10-30 nucleotides in length and have full complementarity with a portion of the antisense strand of a double-stranded Agg-1 promoter gene is determined using the nucleotide sequences shown in FIG. 1.

Such probes or primers are also useful for identifying tissues or cells in which an Agg-1 promoter gene is preferentially expressed or induced either constitutively or at a particular state of tissue differentiation or development or in disease states. Expression of an Agg-1 promoter gene in a particular tissue or group of cells is determined using conventional procedures including, but not limited to, Northern analysis, in situ hybridization to RNA or reverse transcriptase PCR (RT-PCR) amplification. A number of template dependent processes are available to amplify the oligonucleotide sequences present in a given template sample. One of the best known amplification methods is the PCR (referred to as PCR™) which is described in detail in U.S. Pat. Nos. 4,683,195; 4,683,202; and 4,800,159. Other well-known amplification procedures used in the art include RT-PCR [see Sambrook at al., Molecular Cloning: A Laboratory Manual, Second Edition, Cold Spring Harbor Laboratory Press, Cold Spring Harbor, N.Y. (1989)] reverse transcription utilizing thermostable DNA polymerases (WO 90/07641), RT-PCR (see U.S. Pat. No. 5,882,864), the ligase chain reaction (LCR) (see EP 320 308 and U.S. Pat. No. 4,883,750), an oligonucleotide ligase assay (OLA) (see U.S. Pat. No. 5,912,148), QBeta Replicase (see PCT/US87/00880), strand displacement amplification (SDA) (see U.S. Pat. No. 5,916,779) and alternative methods disclosed in U.S. Pat. Nos. 5,843,650; 5,846,709; 5,846,783; 5,849,546; 5,849,497; 5,849,547; 5,858,652; 5,866,366; 5,916,776; 5,922,574; 5,928,905; 5,928,906; 5,932,451; 5,935,825; 5,939,291 and 5,942,391; GS Application No. 2 202 328; and in PCT Application No. PCT/US89/01025, each of which is incorporated herein by reference in its entirety.

Isolated Agg-1 promoter genes are also useful as chromosome markers to map linked gene positions, to identify, chromosomal aberrations, such as translocations, inversions and trisomies, to compare with endogenous DNA sequences in patients to identify potential genetic disorders, and as probes to hybridize and thus discover novel, related promoter genes. For use in such studies and assays, the probes may be labeled with radioisotopes, fluorescent labels or enzymatic labels.

The invention also relates to expression vectors comprising a polynucleotide of the invention. Generally, in addition to the heterologous gene operably-linked to a polynucleotide, the vector will contain at least one eukaryotic marker gene, the appropriate eukaryotic transcriptional and translational stop signals, at least one Shine-Delgarno sequence and initiator codon, a signal that signals polyadenylation of the transcribed messenger RNA (mRNA), and any other DNA sequences necessary or preferred for the appropriate transcription and translation of the heterologous DNA. These additional sequences may include a signal sequence for proteins to be exported or secreted from the host cell and at least one gene for a transcriptional regulator protein. If the vector is used as an extrachromosomal replicating DNA in the eukaryotic cell where it is expressed, the vector will include an origin of replication that functions in the host cell. When the vector is to be integrated into the host chromosomal DNA, it will contain elements necessary to facilitate its integration into the host genome. These elements may be provided by viral vectors such as vaccinia and adenovirus, or by nonviral recombinant plasmids. Preferred expression vectors comprising fragments of the Agg-1 promoter include the binding regions of NFkB and/or STAT3, operably-linked to a heterologous gene encoding a gene product and host cells transformed or transfected with such expression vectors and promoter sequences. Suitable fragments comprise consecutive nucleotides <2000 by and preferably <1000 by and between 100-800 bp.

Host cells provided by this invention expressing heterologous genes under the control of the polynucleotides of this invention can be used to produce proteins, preferably human proteins and fragments thereof. The process involves culturing the transformed cell under conditions wherein the desired protein is expressed, optionally by inducing the activity of the promoter, and purifying the protein from the cell culture. Purification generally involves the steps of cell lysis, homogenization, centrifugation and separation of the desired protein by processes, such as salt fractionation, precipitation and a variety of chromatographic methods such as anion exchange chromatography, hydrophobic interaction chromatography, high resolution chromatography, gel filtration chromatography and the like.

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially- and widely-available.

The insect cell/baculovirus system can produce a high level of protein expression of a heterologous nucleic acid segment, such as described in U.S. Pat. Nos. 5,871,986 and 4,879,236, which are commerically-available. Other examples of expression systems include those which involve a synthetic ecdysone-inducible receptor, or its, pET Expression System, an E. coli expression system, a tetracycline-regulated expression system, an inducible mammalian expression system that uses the full-length CMV promoter and a yeast expression system called the Pichia methanolica Expression System. One of skill in the art would know how to express a vector, such as an expression construct, to produce a nucleic acid sequence or its cognate polypeptide, protein or peptide.

The present invention is related to the identification and characterization of a 5′ flanking region of an Agg-1 promoter gene and regions within the gene that are important for basal promoter activity and/or inducibility. Recently Mizui et al., Mol Biol Rep, Vol. 27, No. 3, pp. 167-173 (2000), have cloned a 1.1 kb 5′ flanking region of the Agg-1 promoter and studied its promoter activity in porcine chondrocytes. The promoter examined by Mizui et al. differs from Agg-1 promoter genes of the present invention by at least 1.3 kb. The data herein agrees with findings that this region has basal promoter activity but further provides mechanisms and regions which are important in regulating functionality of the Agg-1 promoter not identified prior to this disclosure.

Moos at al., J Rheumatol, Vol. 26, No. 4, pp. 870-879 (1999) have shown that an Agg-1 promoter can be potently induced by growth factors. Koshy et al., Arthritis Rheum, Vol. 46, No. 4, pp. 961-967 (2002) have shown that a combination of pro-inflammatory cytokines, such as IL-1β and OSM induce mRNAs for various Aggs and MMPs in chondrocytic cell lines. Understanding that Agg-1 gene expression can be modulated by a number of growth factors and cytokines, experiments are conducted for Agg-1 promoter genes to locate novel binding sites for transcription factors that are known to be activated by cytokines and growth factors. Furthermore, in order to identify control motifs, gene expression cascades and molecular pathways involved in the regulation of Agg-1 promoter transcription, a 2.4 kb fragment-encompassing the 5′ flanking region of the Agg-1 promoter gene has been cloned by Applicants.

The polynucleotides of the present invention comprises multiple transcription factor binding sites including: nuclear factor kappa B (NFkB), signal transducers and activation of transcription (STAT3) and activator protein (AP-1). Referring to FIG. 1 and in the direction 5′ to 3′, an NFkB binding site is located from residue −1800 to residue −1791, a STAT3 binding site located from residue −1842 to residue −1834, both identical with the consensus sequence. An AP-1 site located from residue −2196 to residue to −2190 and an NFkB site located from residue −1448 to residue −1439 has one base pair mismatch compared to the consensus sequence. Interestingly, cartilage-specific cbfa-1 motifs are found in the first intron between residue +659 and residue +1557, SOX9 motif located at +394 and c-krox motif located at residue −575 to residue −567. A genomic fragment representing the promoter region of mouse Agg-1 is also identified utilizing BLAST searches of the mouse genome sequence. Further, the search for specific transcription factor binding sites revealed a very high degree of conservation in the structural organization of the gene between mouse and human.

In another aspect, the invention provides variants of an Agg-1 promoter gene. One means for preparing variants comprises introducing mutations into the polynucleotides of the invention. Such mutants may potentially have enhanced, reduced or altered function relative to the native sequence or alternatively, may be silent with regard to function. Deletion constructs of the invention are obtained by deleting from Agg-1 promoter genes as shown in FIG. 1 those segments shown by Applicants to have negative or positive regulatory activity. FIG. 1 also shows preferred constructs including construct −2.0 kb (SEQ ID NO: 2), construct −1.6 kb (SEQ ID NO: 3), construct −1.2 kb (SEQ ID NO: 4), construct −0.8 kb (SEQ ID NO: 5), construct −0.4 kb (SEQ ID NO: 6) or degenerate variants thereof. The numbering for residue positions used above and elsewhere in the specification refers to the numbering used in FIG. 1 unless stated otherwise. Deletion constructs in which negative regulatory regions are removed result in enhanced promoter activity. Such constructs provide greater sensitivity than the native promoter when used to screen, e.g., for drugs which modulate Agg-1 promoter activity.

Techniques for mutagenizing a DNA segment comprising a specific promoter sequence are well-known to those of skill in the art. Mutagenesis may be performed in accordance with any of the techniques known in the art, such as, and not limited to, synthesizing an oligonucleotide having one or more mutations within the sequence of a particular regulatory region. In particular, site-specific mutagenesis is a technique useful in the preparation of promoter mutants, through specific mutagenesis of the underlying nucleic acid molecule. The technique further provides a ready ability to prepare and test sequence variants, by introducing one or more nucleotide sequence changes into the nucleic acid molecule.

To determine functionality of an Agg-1 promoter gene, various promoter-reporter constructs may be transfected into various host cells. Host cells may be derived from prokaryotes or eukaryotes, depending upon whether the desired result is replication of the vector or expression of part or all of the vector-encoded nucleic acid sequences. Preferred host cells include HEK 293H, SV-40 transformed chondrocytes-C28A2, NIH3T3, human chondrosarcoma-SW1353 and HeLa cells. Fragments of an Agg-1 promoter gene may be generated by PCR primers against the −2.4 kb fragment as a template and detected for luciferase signal transduction. Luciferase values are normalized to 6-Gal values to correct for differences in transfection, efficiencies.

In one aspect, treatment of human articular chondrocytes (HAC) with IL-1β leads to a potent induction of Agg-1, MMP-13 and iNOS mRNAs. Induction of Agg-1 mRNA may be quantitated by real time PCR. All values are normalized to GAPDH. As shown in Table 1, treatment with a combination of IL-1 and OSM leads to a significant induction of Agg-1 promoter mRNA but not from MMP-13 and iNOS mRNA. The induction by PDGF is modest compared to cytokine induction.

TABLE 1

| Cell Treatments | Agg-1 | MMP-13 | INOS |
|---|---|---|---|
| Untreated | 2.3 ± 0.9 | 3.5 ± 1.1 | 5.4 ± 1.8 |
| IL-1 | 160.0 ± 1.4 | 183.0 ± 1.4 | 4500.0 ± 3.2 |
| OSM | 89.0 ± 2.9 | 80.0 ± 1.9 | 478.0 ± 2.1 |
| IL-1 and OSM | 660.0 ± 3.1 | 78.0 ± 2.7 | 270.0 ± 1.9 |
| PDGF | 10.0 ± 1.9 | 12.0 ± 0.9 | 20.0 ± 1.3 |

Agg-1 mRNA is expressed in a wide variety of tissues (see Agg-1 tissue distribution). In Table 2, basal activity is present in all cell lines shown compared to a control sequence, a pGL3 basic vector. The constructs shown in Table 2 are designated according to their location on the 5′ end upstream from the start region (ATG) of an Agg-1 promoter gene. For example, construct-2.4 kb in the promoter region is 2400 base pairs upstream from the start region. The highest basal activity is seen in HEK 293 cells (about 70-fold increase in luciferase activity) and the lowest basal activity for these constructs is found in NIH 3T3 cells (about 5-fold increase in luciferase activity). Mizul et al. (2000), supra, have reported that the NF-1 (−847) binding site is involved in the negative regulation of Agg-1 expression utilizing their disclosed promoter gene. In agreement with Mizui et al. (2000), supra, a modest repressive effect of the luciferase activity is found in Agg-1 promoter constructs disclosed herein which carry the NF-1 site located −847 by upstream of ATG. Agg-1 promoter constructs encompassed by the invention and located in the 5′ end upstream from the start region include the regions between −1 to −0.4 kb (SEQ ID NO: 6), −1 to −0.8 kb (SEQ ID NO: 5), −1 to −1.2 kb (SEQ ID NO: 4) −1 to −1.6 kb (SEQ ID NO 3), −1 to −2.0 kb (SEQ ID NO: 2) and −1 to −2.4 kb (SEQ ID NO: 1). Preferred construct include the −1 to −0.8 kb (SEQ ID NO: 5) construct which has the highest basal promoter activity in the cell lines tested and the −1 to −24 kb (SEQ ID NO: 4) which encompasses the inducible region.

TABLE 2
Basal Activity of the Agg-1 Promoter

| | Basal Promoter Activity | | | | |
|---|---|---|---|---|---|
| | Fold Increase in Luciferase Activity over pGL3 Basic Vector | | | | |
| Agg-1 Constructs | C28A2 | SW1353 | NIH 3T3 | HeLa | 293H |
| pGL3 basic vector | 1.0 ± 0.2 | 1.0 ± 0.1 | 1.0 ± 0.4 | 1.0 ± 0.1 | 1.0 ± 0.3 |
| −2.4 kb Agg-1 Luc | 35.0 ± 3.5 | 24.0 ± 2.6 | 7.3 ± 0.9 | 14.0 ± 2.3 | 59.0 ± 4.6 |
| −2.0 kb Agg-1 Luc | 29.0 ± 4.2 | 20.0 ± 3.5 | 6.0 ± 0.9 | 15.0 ± 1.2 | 61.0 ± 2.0 |
| −1.6 kb Agg-1 Luc | 29.0 ± 1.7 | 28.0 ± 3.2 | 6.0 ± 0.6 | 14.0 ± 2.0 | 44.0 ± 4.4 |
| −1.2 kb Agg-1 Luc | 30.0 ± 6.3 | 28.0 ± 3.1 | 6.0 ± 0.8 | 15.0 ± 1.5 | 68.0 ± 5.8 |
| −0.8 kb Agg-1 Luc | 32.5 ± 2.8 | 37.0 ± 2.9 | 8.0 ± 0.4 | 18.5 ± 2.3 | 71.0 ± 1.7 |
| −0.4 kb Agg-1 Luc | 4.0 ± 1.2 | 9.0 ± 0.7 | 5.0 ± 1.2 | 3.0 ± 1.2 | 1.5 ± 2.2 |

### Inducibility of the Agg-1 Promoter Gene by Cytokines and Growth Factors

In contrast to the ubiquitous nature of Agg-1 basal activity, inducibility of an Agg-1 promoter genes is more specific. Various promoter-reporter constructs may be transfected into HEK 293H, SV-40 transformed chondrocytes-C28A2, NIH 3T3, human chondrosarcoma SW1353 and HeLa cells which are induced with and OSM, 48 hours post-transfection. Table 3 shows data from multiple experiments after correction of the luciferase values for transfection efficiencies. The inducibility value, as determined by luciferase activity, is highest in SW1353 cells (about 10-fold) and HeLa cells (about 35-fold) compared to basal promoter activity represented by pGL3 basic vector. Luciferase activity is not detected in the other cell lines tested. The induction of luciferase activity by a combination of IL-1β and OSM (10-fold for SW1353 and 35-fold for HeLa cells) is much higher than the additive effect of each of the single cytokines (which is about 2- to 3-fold for SW1353 and 5-fold for HeLa cells). Although not wishing to be bound by theory, the data suggests that there could be a cooperative interaction between the transcription factors activated by these cytokines in the Agg-1 promoter gene which leads to the potent induction of Agg-1 promoter transcription.

TABLE 3
Delineation of the Inducible Region of the Agg-1 Promoter

| | Induced Promoter Activity | | | | |
|---|---|---|---|---|---|
| | Fold Increase in Luciferase Activity over pGL3 Basic Vector | | | | |
| Agg-1 Constructs | C28A2 | SW1353 | NIH 3T3 | HeLa | 293H |
| pGL3 basic vector | 1.0 ± 0.2 | 1.6 ± 0.4 | 1.1 ± 0.1 | 4.0 ± 0.1 | 0.8 ± 0.3 |
| −2.4 kb Agg-1 Luc | 1.2 ± 0.1 | 10.2 ± 1.9 | 2.0 ± 0.1 | 34.0 ± 2.3 | 0.7 ± 4.6 |
| −2.0 kb Agg-1 Luc | 1.3 ± 0.2 | 7.5 ± 1.0 | 1.9 ± 0.1 | 36.0 ± 1.2 | 0.9 ± 2.0 |
| −1.6 kb Agg-1 Luc | 1.3 ± 0.1 | 6.8 ± 2.0 | 1.6 ± 0.2 | 19.0 ± 2.0 | 0.9 ± 4.4 |
| −1.2 kb Agg-1 Luc | 1.2 ± 0.2 | 3.7 ± 1.0 | 1.3 ± 0.1 | 17.0 ± 1.5 | 0.9 ± 5.8 |
| −0.8 kb Agg-1 Luc | 1.0 ± 0.1 | 4.8 ± 1.9 | 1.4 ± 0.1 | 18.0 ± 2.3 | 1.0 ± 1.7 |
| −0.4 kb Agg-1 Luc | 1.0 ± 0.1 | 1.3 ± 0.3 | 1.1 ± 0.1 | 7.0 ± 1.2 | 1.1 ± 2.2 |

Various binding regions comparable to Agg-1 promoters have been identified which are activated by inducers used in the invention. For MMP-13, several reports [see Tardif et al., Biochem J, Vol. 323, Part 1, pp. 13-16 (1997); Pendas et al., Genomics, Vol. 40, No. 2, pp. 222-233 (1997); and Li, Dehnade and Zafarullah, J Immunol, Vol. 166, No. 5, pp. 3491-3498 (2001)], which disclose that the AP-1 site in the promoter region confers responsiveness to a variety of inducers of the JAK/STAT signaling pathway. However, the STAT binding sites in the MMP-13 promoter have not been characterized for functionality to date. The human iNOS promoter contains an overlapping bifunctional NFkB-STAT1 motif that binds either NFkB or STAT1 in response to cytokines. Based on the results identified herein, activation of STAT1 signaling region in the iNOS promoter may serve as a positive or negative regulator of human iNOS transcription, depending on the cell type. See Ganster et al., Proc Natl Acad Sci USA, Vol. 98, No. 15, pp. 8638-8643 (2001); and Yu, Zhang and Kone, Biochem J, Vol. 367, Part 1, pp. 97-105 (2002).

Applicants have shown that cytokine treatment of HAC cells leads to simultaneous induction of Agg-1 promoter, MMP-13 and iNOS mRNAs. In HAC, simultaneous activation of NFkB and STAT signaling pathways by treatment with a combination of IL-1 and OSM results in a significant or super induction of the Agg-1 mRNA which is 3- to 4-fold greater activity than treatment with either IL-1 or OSM alone (see Table 1).

Other assays may be used to identify responsive elements in a promoter region or gene. Such assays will be known to those of skill in the art [see, e.g., Sambrook et al. (1989), supra; Zhang et al. (1997); Shan et al. (1997); Dal and Burnstein (1996); Cleutjens et al. (1997); Ng et al. (1994); and Shida et al. (1993)], and include DNase I footprinting studies, Elecromobility Shift Assay patterns (EMSA), the binding pattern of purified transcription factors, effects of specific transcription factor antibodies in inhibiting the binding of a transcription factor to a putative responsive element, Western analysis, nuclear run-on assays and DNA methylation interference analysis.

In another aspect. EMSA using nuclear extracts from control- and cytokine-treated HAC, SW1353 or HeLa cells reveals that NFkB, STAT3 and AP-1 nuclear protein complexes are enhanced in cytokine treated extracts. EMSAs may be performed with nuclear extracts prepared from IL-1- and OSM-treated HAC, SW1353 or HeLa cells. The Agg-1 promoter site specific NFkB (−1800), AP-1 (−2196) and STAT3 (−1842) oligonucleotides may be used for the study. The extracts from cytokine-treated cells induce strong gel shift complexes for NFkB, AP-1 and STAT3. These complexes are not detected with nuclear extracts from untreated samples. To demonstrate specificity of binding, 50-fold molar excess of different unlabelled probes may be used as competitors for binding. Unlabeled NFkB, AP-1 and STAT3 elements completely block the binding whereas mutant oligonucleotides do not compete for binding. Gel shifts with NFkB (−1448) oligonucleotide from the promoter sequence lead to a much weaker complex formation. This data suggests that NFkB and STAT3 activation is crucial for the induction of a human Agg-1 promoters by transcription factors.

Supershift analysis of AP-1 bands in the presence of specific antibodies to c-Jun, JunB, JunD, c-Fos, FosB, Fra 1 and 2, CREB 1 and 2/ATF 1, 2 and 3 may also be carried out in order to identify which among these factors contributes to the formation of AP-1 complex. The results suggest that c-Jun and JunB are the main components of the AP-1 complex in IL-1- and OSM-treated nuclear extracts. Similar supershift analysis with NFkB-p50 and NFkB-p65 antibodies and STAT 1, 3 and 5 antibodies in IL-1- and OSM-treated extracts suggest that the main component of the STAT complex is STAT3 and that of NF-kB are the p-50 and p-65 subunits.

In order to identify which sites in the Agg-1 promoter are important for cytokine inducibility, mutagenesis may be carried out on the −2.4 kb Agg-1 promoter construct to selectively mutate the NFkB (−1448, −1800), AP-1 (−2196) and STAT3 (−1842) regions separately or in combinations of at least two. SW1353 or HeLa cells may be stimulated with IL-1 and OSM following transient transfection of the constructs (see Table 3).

The data in Table 4 represents multiple experiments wherein the luciferase values are normalized to β-Gal values to correct for transfection efficiencies. Table 4 indicates that NFkB (−1800) and STAT3 (−1842) are critical for IL-1- and OSM-mediated induction of the Agg-1 promoter whereas the AP-1 (−2196) and NFkB (−1448) sites do not produce as significant results for induction as NFkB (−1800) and STAT3 (−1842) individually or in combination. The STAT3 mutation is the most detrimental in producing promoter activity induced by IL-1β and OSM. Double mutation of NFkB and STAT3 sites further reduce induction with IL-1β and OSM. Other double mutants which include the AP-1 site do not diminish IL-1β- and OSM-mediated induction significantly.

Thus, the EMSA data confirms that the region between −1.2 kb and −2.4 kb which encompasses the NFkB, STAT and AP-1 sites are important for inducibility. The EMSA data taken together with the mutagenesis data suggest that the proximal NFkB (−1800) and STAT3 (−1842) sites can act co-operatively to superinduce the promoter (see Tables 3 and 4). Without being bound by theory, it is proposed that the occupancy of both these sites by the respective transcription factors (NFkB and STAT3) can facilitate binding of a third factor. The invention described herein provides the first instance of data that indicates cross talk occurs between NFkB and STAT3 signaling pathways which has a synergistic effect on the regulation of human Agg-1 gene expression by cytokines.

TABLE 4
Site Directed Mutational Analysis of the Agg-1 Promoter

| | | | | |
|---|---|---|---|---|
| Agg-1 Constructs (2.4 kb) | Uninduced | IL-1 | OSM | IL-1 and OSM |

SW1353 Cells (fold increase in luciferase activity over pGL3 basic vector)

| | | | | |
|---|---|---|---|---|
| pGL3 basic vector | 1.0 | 1.0 ± 0.10 | 1.0 ± 0.10 | 1.0 ± 0.01 |
| Agg-1 wild-type | 1.0 | 1.3 ± 0.12 | 6.8 ± 1.00 | 10.4 ± 0.50 |
| NFkB (−1448) mutant | 1.0 | 1.2 ± 0.14 | 5.9 ± 0.15 | 9.0 ± 0.10 |
| NFkB (−1800) mutant | 1.0 | 1.3 ± 0.09 | 2.9 ± 0.10 | 4.2 ± 0.90 |
| STAT3 (−1842) mutant | 1.0 | 1.4 ± 0.13 | 1.6 ± 0.15 | 2.1 ± 0.15 |
| AP-1 (−2196) mutant | 1.0 | 1.6 ± 0.15 | 5.4 ± 0.10 | 8.9 ± 0.40 |
| NFkB (−1800)/STAT3(−1842) | 1.0 | 1.1 ± 0.14 | 1.6 ± 0.05 | 1.4 ± 0.05 |
| double mutant | | | | |
| NFkB (−1800)/AP-1 (−2196) | 1.0 | 1.6 ± 0.15 | 3.2 ± 0.09 | 4.1 ± 0.50 |
| double mutant | | | | |
| STAT3 (−1842)/AP-1 (−2196) | 1.0 | 1.4 ± 0.11 | 2.4 ± 0.10 | 2.2 ± 0.10 |
| double mutant | | | | |

HeLa Cells (fold increase in luciferase activity over pGL3 basic vector)

| | | | | |
|---|---|---|---|---|
| pGL3 basic vector | 1.0 | 1.0 ± 0.01 | 1.9 ± 0.10 | 1.6 ± 0.01 |
| Agg-1 wild-type | 1.0 | 1.5 ± 0.05 | 16.8 ± 0.80 | 35.0 ± 2.10 |
| NFkB (−1448) mutant | 1.0 | 1.4 ± 0.04 | 15.1 ± 0.50 | 30.0 ± 0.15 |
| NFkB (−1800) mutant | 1.0 | 1.4 ± 0.09 | 11.2 ± 2.50 | 22.8 ± 2.20 |
| STAT3 (−1842) mutant | 1.0 | 1.4 ± 0.08 | 10.5 ± 0.90 | 16.5 ± 0.95 |
| AP-1 (−2196) mutant | 1.0 | 1.6 ± 0.09 | 14.4 ± 1.50 | 31.3 ± 1.20 |
| NFkB (−1800)/STAT3 (−1842) | 1.0 | 1.2 ± 0.05 | 9.8 ± 0.80 | 13.2 ± 0.90 |
| double mutant | | | | |
| NFKB (−1800)/AP-1 (−2196) | 1.0 | 1.5 ± 0.10 | 13.1 ± 1.20 | 19.0 ± 1.10 |
| double mutant | | | | |
| STAT3 (−1842)/AP-1 (−2196) | 1.0 | 1.4 ± 0.07 | 11.9 ± 1.10 | 16.0 ± 1.00 |
| double mutant | | | | |

It has been reported that cbfa1 has a crucial role in the transcription of MMP-13 gene. See Jimenez et al., Mol Cell Biol, Vol. 19, No. 6, pp. 4431-4442 (1999). The Agg-1 promoter gene of the present invention is analyzed for cbfa1 sites and interestingly, 3 sites are present in the first intron of the Agg-1 promoter gene between residues +633 and +1746. Other sites identified in the Agg-1 promoter gene of the invention includes c-krox (residues −575 to −567), SOX9 (residues +394 to +400) and IRS/FKHRL (residues −928 to −921) sites. Ghayor et al., J Biol Chem, Vol. 275, No. 35, pp. 27421-27438 (2000) have shown that there are c-krox binding sites in the first intron of the COL2A1 gene and that the c-krox transcription factor is crucial for chondrocyte specific transcription of the COL2A1 gene. The role of cbfa1, c-krox, SOX9 and IRS/FKHRL transcription factors in the regulation of Agg-1 gene transcription needs to be investigated further.

The present invention also provides regulatory sequences present in human genomic DNA, which can be used to target tissues or cells in which Agg-1 promoter activity is found. The polynucleotides of the invention may be used for diagnostic purposes. The polynucleotides may also be used to detect and quantify Agg-1 promoter activity or expression of gene transcripts in biopsied tissues in collagen and/or chondrocyte degrading enzymes are correlated with a disease. The diagnostic assay may also be used to determine whether certain therapeutic agents modulate Agg-1 promoter activity.

In general, gene expression may be determined by measuring the production of RNA, protein or both. A gene product (RNA or protein) may be isolated and/or detected by methods well known in the art. Following detection, one may compare the results seen in a given cell line or individual with a statistically significant reference group of non-transformed control cells. Alternatively, one may compare production of RNA or protein products in cell lines transformed with the same gene operably-linked to various mutants of a promoter sequence. In this way, it is possible to identify regulatory regions within a novel promoter sequence by their effect on the expression of an operably-linked gene.

In Tables 2 and 3, the Agg-1 promoter was shown to be induced in various cells. Thus this invention also provides methods and compositions for regulating expression of heterologous DNA to treat or detect diseases involving an Agg-1 promoter activity or expression of proteins which bind to Agg-1 promoters of the invention. Such diseases may also involve enhanced expression or activity of one or more transcriptional regulators which may directly or indirectly induce expression of Agg-1, such as cytokines or growth factors. Specifically, the invention has utility in the Study of and therapy for diseases involving inflammation, e.g., several neurological and respiratory disorders, rheumatoid arthritis, osteoarthritis or non-inflammatory arthritis and in tissues where an Agg-1 promoter is over-expressed.

The present invention also relates to nucleic acid molecules containing at least part of the sequence of an Agg-1 promoter gene of the present invention described above (including its variants and derivatives). Such nucleic acid molecules can competitively inhibit the binding between an Agg-1 promoter and proteins, e.g., transcription factors, that can bind to it, regardless of whether or not the nucleic acid molecule has promoter activity. Consequently, if the nucleic acid molecule competes for binding a protein that inhibits promoter activity, the promoter activity can be enhanced in this way. On the other hand, if the nucleic acid molecule competes with the binding site for a protein that enhances the promoter activity, the promoter activity can be inhibited in this way. Such methods may be used to screen for transcriptional modulators to develop novel therapeutic agents depending on the desired promoter activity. Further, such methods using the Agg-1 promoter gene of the invention may also be used to screen for novel genes or pathways that are involved in the coordinate induction of the promoter gene described herein.

In another aspect, the polynucleotides and derivatives of the invention are also useful in inhibiting proteolytic degradation in chondrocytic and nonchondrocytic cells, screening for drugs that selectively modulate transcription of the polynucleotides in chondrocyte cells and drugs that modulate the degradation of articular cartilage.

In one aspect, this invention provides methods for modulating articular cartilage degradation in a mammal subject to or predisposed to having arthritic conditions, such as rheumatoid arthritis or osteoarthritis with an expression vector comprising novel promoter sequences of the invention. The invention further provides a use of an expression vector comprising novel promoter sequences of the invention in the preparation of a medicament for modulating articular cartilage degradation in a mammal subject to or predisposed to having arthritic conditions. Agg-1 promoters of the invention may be mutated then operably-linked to a gene that encodes for a proteoglycan thereby inhibiting gene expression of gene products. Mutated Agg-1 promoters may also prevent binding of cytokines to binding sites within the Agg-1 promoter binding regions. Point mutations and/or deletions of the NFkB and/or STAT3 sites will inhibit Agg-1 promoter activity thereby preventing expression of Aggrecanase enzymes that contribute to cartilage degradation.

Screenable markers constitute another efficient means for quantifying the expression of a given gene. Potentially any screenable marker could be expressed and the marker gene product quantified, thereby providing an estimate of the efficiency with which the promoter directs expression of the gene. Quantification can readily be carried out using either visual means, or, e.g., a photon counting device.

Agents which block Agg-1 promoter induction or activity include antibodies, antisense-RNA, ribozyme, hormones, proteins or polypeptides. Such agents may be administered to a subject by in vivo or ex vivo transfection. Ex vivo methods used in the art may be used and generally involve explanting cells from the mammal, transfecting the cells with an expression vector comprising a nucleic acid comprising an Agg-1 promoter operatively-linked to genes coding for a desired molecule and selecting and reimplanting into the mammal cells which have incorporated and express the heterologous nucleic acid sequence. In vivo transfection can be accomplished by methods used in the art, such as direct injection of the above expression vectors.

The host cells transformed with the novel promoter sequences of this invention can also be used to identify compounds which specifically bind the Agg-1 promoter and act as agonists or antagonists of the Agg expression or activity. As described earlier, deletion constructs of Agg-1 promoters can be employed to increase or decrease its ability to drive expression of gene products and thereby increase the sensitivity of the assay.

### Antibodies

In another aspect, the present invention relates to antibodies which are specific for and bind to immunogenic regions in an Agg-1 promoter gene. Such antibodies are useful research tools for identifying tissues that contain elevated levels of proteins produced by induction of an Agg-1 promoter and for purifying the respective protein from cell or tissue extracts, medium of cultured cells or partially purified preparations of intracellular and extracellular proteins by affinity chromatography. Such antibodies are also useful for identifying and diagnosing diseases associated with elevated or reduced levels of proteins produced by Agg-1 promoter activity. Such antibodies are also useful for monitoring the effect of therapeutic agents on the synthesis, induction and secretion of Agg-1 promoters by cells in vitro and in vivo. Such antibodies may also be employed in procedures, such as co-immunoprecipitation and co-affinity chromatography, for identifying other proteins, activators and inhibitors of an Agg-1 promoter gene.

The present invention also provides a method for detecting Agg-1 promoter activity or a protein expressed by induction of an Agg-1 promoter in a bodily sample from a patient using antibodies immunospecific for Agg-1 promoter genes or gene products. The method comprises contacting the antibody with a sample taken from the patient; and assaying for the formation of a complex between the antibody and the corresponding Agg-1 promoter gene, immunogenic fragments or gene products thereof present in the sample. The sample may be a tissue or a biological fluid, including but not limited to whole blood, serum, synovial fluid, stool, urine, breast, cerebrospinal fluid, semen, diagnostic washes from trachea, stomach and other bowel segments, tissue biopsies or excised tissue, cells obtained from swabs and smears. To monitor changes in expression of the Agg-1 promoter during joint disorders, the preferred sample is synovial fluid. To monitor changes in expression of Agg-1 promoter during inflammation the preferred samples include, but are not limited to, serum, body fluids or biopsy tissue.

The sample may be untreated, or subjected to precipitation, fractionation, separation or purification before combining with an anti-Agg-1 promoter antibody. For ease of detection, it is preferred that isolated proteins from the sample be attached to a substrate, such as a column, plastic dish, matrix or membrane, preferably nitrocellulose. Preferably, the detection method employs an enzyme-linked immunosorbent assay (ELISA) or a Western immunoblot procedure.

Interactions between an Agg-1 promoter in the sample and the corresponding anti-Agg-1 promoter antibody are detected by radiometric, calorimetric or fluorometric means, size-separation or precipitation. Preferably, detection of an antibody-Agg-1 promoter complex is by addition of a secondary antibody that is coupled to a detectable tag, such as e.g., an enzyme, fluorophore or chromophore. Formation of the complex is indicative of the presence or activity of an Agg-1 promoter in the test sample. Thus, the method is used to determine whether there is a decrease or increase in Agg-1 promoter activity in a test sample as compared to levels of the promoter protein in a control sample and to quantify the amount of induction of the promoter or expression of a protein in the test sample. Deviation between control and test values establishes the parameters for diagnosing the disease.

### Drug Screening

By using an Agg-1 promoter gene of the present invention, it is also possible to screen compounds, as well as nucleic acid molecules and proteins that regulate the activity of Agg-1 promoters or the gene encoding it. Thus, the present invention also relates to a method of screening compounds that regulate Agg-1 promoter activity. One embodiment of the screening method comprises the steps of bringing a test sample into contact with an Agg-1 promoter in the presence of a test compound and selecting a compound which promotes or inhibits the binding between Agg-1 promoter genes of the present invention and a protein in the test sample. For example, the method can be carried out by binding a cell nuclear extract to an Agg-1 promoter which has been labeled with an isotope and the like, subjecting the binding product to polyacrylamide gel electrophoresis, and detecting, by gel shift assay, the band representing the complex between the protein in the nuclear extract and an Agg-1 promoter gene of the present invention. Upon adding a DNA probe, the test compound is also added to select a compound that enhances or inhibits the formation of the bands representing the complex between the protein in the nuclear extract and the promoter of the present invention. This method enables screening of compounds that directly act on an Agg-1 promoter and compounds that act on the proteins binding to an Agg-1 promoter of the present invention. If, e.g., a protein that binds to Agg-1 promoter genes of the present invention inhibits the activity of the promoter in vivo, compounds that inhibit the binding between a protein and an Agg-1 promoter gene of the present invention would enhance Agg-1 promoter activity. Furthermore, if a protein that binds to Agg-1 promoters of the present invention has already been isolated, then it is also possible to use a recombinant protein of the protein in place of the cell nuclear extract.

For screening purposes an appropriate host cell that expresses an Agg-1 promoter, preferably a chondrocyte cell, more preferably a human articular chondrocyte cell, is transformed with an expression vector comprising a reporter gene operably-linked to an Agg-1 promoter of the invention. The transformed host cell is exposed to various test substances and then analyzed for expression of the reporter gene. This expression can be compared to expression from cells that were not exposed to the test substance. A compound which increases the induction, transcription or activity of an Agg-1 promoter will result in increased reporter gene expression relative to the control. Similarly, compounds which act as antagonists for Agg-1 promoter signaling pathways will result in decreased reporter gene expression relative to the control.

Transformed cells may be induced with a transcriptional inducer, such as cytokines or growth factors. Preferred cytokines include IL-1 or OSM. Transcriptional activity is measured in the presence or absence of a pharmacologic agent of known activity, i.e., a standard agent, or putative activity, i.e., a test agent. A change in the level of expression of the reporter gene in the presence of the test agent is compared to that effected by the standard agent. In this way, the ability of test agents to affect Agg-1 promoter transcription and their relative potencies can be determined.

Preferred reporter genes are those which produce a protein product that is easily measured in a routine assay. Suitable reporter genes include, but are not limited to, chloramphenicol acetyl transferase, luciferase and β-galactosidase. Convenient assays include calorimetric, fluorimetric and enzymatic assays. Most preferred are reporter genes that are expressed within the cell and whose extracellular products are directly measured in the intracellular medium, or in an extract of the intracellular medium of a cultured cell line. This provides advantages over using a reporter gene whose product is secreted, since the rate and efficiency of the secretion introduce additional variables which complicate interpretation of the assay.

Thus in one aspect of the invention one can screen for test compounds that regulate the activity of an Agg-1 promoter by:
(a) contacting a host cell in which an Agg-1 promoter gene disclosed herein is operably-linked to a reporter gene with a test medium containing the test compound under conditions which allow for expression of the reporter gene;
(b) measuring the expression of the reporter gene in the presence of the test medium;
(c) contacting the host with a control medium which does not contain the test compound but is otherwise identical to the test medium in (a), under conditions identical to those used in (a);
(d) measuring the expression of reporter gene in the presence of the control medium; and
(e) relating the difference in expression between (b) and (d) to the ability of the test compound to regulate the activity of said Agg-1 promoter.

Another aspect of the invention is its use in screening for pharmacologically active test agents that modulate Agg-1 promoter activity, particularly among the transcription factor binding sites; such as AP-1, NFkB and STATS, either by affecting signal transduction pathways that necessarily precede transcription or by directly affecting transcription of a Agg-1 promoter genes. The use of test agents may be used to identify drugs, polypeptides, auto-antibodies or other compounds which bind to Agg-1 promoters, preferably specifically to binding regions within an Agg-1 promoter, and gene products produced therefrom with sufficient affinity to block or facilitate promoter activity. The activity of Agg-1 promoters may be assayed in the presence or absence of a putative inhibitor using any of a variety of assays known in the art and also exemplified below. To detect promoter activity or to monitor the extent of expression as a result of induction or transcription of an Agg-1 promoter, a substrate is tagged in a manner which provides a detectable signal upon expression of a protein product. For example, the substrate may be tagged with a detectable label used in the art.

Thus in another aspect of this invention provides methods of measuring the ability of a test compound to modulate Agg-1 promoter gene transcription by
(a) contacting a host cell in which an Agg-1 promoter gene disclosed herein is operably-linked to a reporter gene with an inducer of Agg-1 promoter activity under conditions which allow for expression of the reporter gene;
(b) measuring the expression of the reporter gene in the absence of the test compound;
(c) exposing the host cells to the test compound either prior to, simultaneous with, or after contacting, the host cells with the inducer,
(d) measuring the expression of the reporter gene in the presence of the test compound; and
(e) relating the difference in expression between (b) and (d) to the ability of the test compound to modulate Agg-1 promoter gene transcription.

Since different inducers are known to affect different modes of signal transduction, e.g., cAMP responsive, calcium ion responsive, it is thus possible to identify with greater specificity compounds which affect a particular signal transduction pathway. Furthermore, since an Agg-1 promoter gene is shown to be super induced with cytokine treatment, assays measuring its induction may provide a means of identifying compounds which will inhibit and/or reverse specific signal transduction.

In a another aspect, the invention provides a method of determining the presence of articular cartilage degenerative conditions in a subject. The method includes providing a sample from the subject and measuring the amount of polynucleotides selected from the group consisting of SEQ ID NOs: 1-6, in the subject sample. The amount of Agg-1 polynucleotides in the subject sample is then compared to the amount of Agg-1 polynucleotides in a control sample from a subject. An alteration in the amount of Agg-1 polynucleotide or transcription in the subject sample relative to the amount of Agg-1 polynucleotide or transcription in the control sample indicates the subject has articular cartilage degenerative conditions. A control sample is preferably taken from a matched individual, i.e., an individual of similar age, sex or other general condition but who is not suspected of having articular cartilage degeneration or upregulation of proteglycan. Alternatively, the control sample may be taken from the subject at a time when the subject is not suspected of having articular cartilage degeneration.

In a further aspect, the invention provides a method of determining predisposition to arthritis, rheumatoid arthritis or osteoarthritis, in a subject. The method includes providing a nucleic add sample, e.g., RNA or DNA, or both, from the subject and measuring the amount of the Agg-1 promoter polynucleotides expressed in the subject nucleic acid sample. The amount of Agg-1 promoter polynucleotides in the subject nucleic acid is then compared to the amount of Agg-1 promoter polynucleotides in a control sample. An alteration in the amount of Agg-1 nucleic acid in the sample relative to the amount of Agg-1 in the control sample indicates the subject has a predisposition to arthritis.

In a still further aspect, the invention provides method of treating or preventing or delaying an Agg-1 associated disorder. The method includes administering to a subject in which such treatment or prevention or delay is desired an inhibitor of transcription of Agg-1 promoter genes in an amount sufficient to treat, prevent or delay Agg-1 associated disorders in the subject.

in another aspect, the invention provides a use of an inhibitor of transcription of Agg-1 promoter genes in the preparation of a medicament for treating, preventing or delaying Agg-1 associated disorders in a subject in which such treatment or prevention or delay is desired.

### EXAMPLES

In practicing the present invention, many conventional techniques in molecular biology, microbiology and recombinant DNA are used. These techniques are well-known and are explained in, e.g., Current Protocols in Molecular Biology, Vols. I-III, Ausubel, Ed. (1997); Sambrook et al. (1989), supra; DNA Cloning: A Practical Approach, Vols. I and II, Glover, Ed. (1985); Oligonucleotide Synthesis, Gait, Ed. (1984); Nucleic Acid Hybridization, Hames and Higgins, Eds. (1985); Transcription and Translation, Names and Higgins, Eds. (1984); Animal Cell Culture, Freshney, Ed. (1986); Immobilized Cells and Enzymes, IRL Press (1986); Perbal, A Practical Guide to Molecular Cloning; the series, Methods in Enzymology, Academic Press, Inc. (1984); Gene Transfer Vectors for Mammalian Cells, Miller and Calos, Eds., Cold Spring Harbor Laboratory, NY (1987); and Methods in Enzymology Vols. 154 and 155, Wu and Grossman, and Wu, Eds., respectively (1987).

### Example 1

### Induction of Agg-1 Promoter Gene in Cells

HAC SW1353 or HeLa cells [ATCC] are grown to confluency in 24-well plates in DMEM high glucose medium containing penicillin, streptomycin and fungizone (invitrogen, Carlsbad, Calif.). The cells are serum starved overnight and induced with IL-1β (10 ng/mL) (Peprotech, Rockyhill, N.J.) alone or with Oncostatin M (50 ng/mL) (R&D systems, Minneapolis, Minn.) or PDGF (50 ng/mL) (R&D systems, Minneapolis, Minn.) for 16 hours.

### Example 2

### RNA Isolation and Real Time PCR

Total RNA is isolated using Rneasy Mini kit (Qiagen, Valencia, Calif.) according to the protocol described by the manufacturer. The on-column DNase I digestion is employed to eliminate contaminating genomic DNA. First-strand cDNA is synthesized using random primers with the High-Capacity cDNA Archive kit (PE Applied Biosystems, Foster City, Calif.) in a 100 μL reaction volume. Real time PCR is performed in a 384-well format on the ABI Prism 7900HT Sequence Detection System (PE Applied Biosystems) using SYBRGreen PCR Master Mix (PE Applied Biosystems).

The cDNA template and PCR mix are distributed using the Biomek FX liquid handling robot (Beckman Coulter, Fullerton, Calif.). The 20 μL reaction will contain 5 μL cDNA, 200 nM forward and reverse primers and SYBRGreen PCR Master Mix. The default cycling program is followed by a dissociation stage whereby a melting curve is generated to confirm the specificity of the PCR product and the absence of primer dimers. Primers for RT-PCR are designed with Primer Express software (PE Applied Biosystems) under default parameters and reaction conditions and their sequences are as follows:

Aggrecanase-I forward

| | |
|---|---|
| 5′-TTTCCCTGGCAAGGACTATGA-3′ | (SEQ ID NO: 7) |
| Aggrecanase-I reverse | |
| 5′-AATGGCGTGAGTCGGGC-3′ | (SEQ ID NO: 8) |
| MMP-13 forward | |
| 5′-TGATCTCTTTTGGAATTAAGGAGCAT-3′ | (SEQ ID NO: 9) |
| MMP-13 reverse | |
| 5′-ATGGGCATCTCCTCCATAATTTG-3′ | (SEQ ID NO: 10) |
| iNOS forward | |
| 5′-GCAAACCTTCAAGGCAGCC-3′ | (SEQ ID NO: 11) |
| iNOS reverse | |
| 5′-TGCTGTTTGCCTCGGACAT-3′ | (SEQ ID NO: 12) |

### Example 3

Identification of Agg-1 Promoter Gene

A 300 by sequence including the ATG codon of the human Agg-1 cDNA clone NM_{—}005099) is used to BLAST the human genome assembly sequence. See The International Human Genome Sequencing Consortium, Nature, Vol. 409, pp. 860-921 (2001). A 10 kb upstream region is identified (NT 004668) and this sequence is used for cloning different regions of the promoter.

### Identification of Transcription Factor Binding Sites in Agg-1 Promoter Gene

The consensus sequences of transcription factors are used to search potential binding sites in the Agg-1 promoter using an in-house search program. Sequences with perfect match to NFkB and STAT3 consensus sequences are located −1800 and −1842 by upstream of the initiation codon, ATG. An AP-1 site and a second NFkB site with one base pair mismatch to the consensus sequence are located at −2196 by and −1448 bp, respectively. These sites are known to be activated by cytokines and growth factors. Other sites like c-krox and cbfa1, which are known to be cartilage specific, are also identified down stream of the ATG.

### Cloning of a 2.4 KB 5′ Flanking Region of Agg-1 Promoter Gene.

A DNA segment representing 2.4 kb upstream of the ATG (−2.4 kb), the promoter region of the invention, is isolated by PCR using human genomic DNA (Clontech, Palo Alto, Calif.) as template. The nucleotide sequence of primers used for the PCR reaction are given below. The primers carry restriction endonuclease cleavage sites (Xho1 site in the 5′ oligo and Hind III in the 3′ oligo) that can be used for subsequent subcloning into the pGL3 basic luciferase reporter plasmid (Promega, Madison, Wis.).

The PCR cycling conditions for the amplification are as follows:

Denaturation 30 seconds at 95° C., annealing 40 seconds at 60° C., extension for 2.5 minutes at 68° C. PCR reactions are performed using the expand long template PCR amplification system (Roche Diagnostics, Indianapolis, Ind.) for 30 cycles. The PCR product is purified by agarose gel electrophoresis, digested with Xho I and Hind III and subcloned into the pGL3 basic reporter plasmid, which is linearized with Xho I and Hind III.

### Example 4

### Generation of Deletion Constructs of Agg-1 Promoter Gene

PCR primers are designed to generate constructs encompassing −400 bp, −800 bp, −1.2 kb, −1.6 kb and −2.0 kb of the promoter region. The constructs are generated by PCR using the −2.4 kb fragment as a template and PCR conditions described above. The nucleotide sequence of the primers used to generate the constructs are given below. Xho I (CTCGAG) and Hind III (AAGCTT) restriction sites are underlined:

### PCR Primers

Forward (−2400)
(SEQ ID NO: 13)
5;-GCGCGCTCGAGCTGCATTTATTTGCCTTGATCC-3′
Reverse (−1)
(SEQ ID NO: 14)
5′-GCGCGAAGCTTGGCACTGGTACTGCAGCTGGGA-3′
Forward (−2000)
(SEQ ID NO: 15)
5′-GCGCGCTCGAGGTGGGTGATCCAGGAAGTGATA-3′
Forward (−1600)
(SEQ ID NO: 16)
5′-GCGCGCTCGAGGATTTCTCCCAGTACCCTAATTTCC-3′
Forward (−1200)
(SEQ ID NO: 17)
5′-GCGCGCTCGAGTAGTGGTAACTCAGGAAGGGGG-3′
Forward (−800)
(SEQ ID NO: 18)
5′-GCGCGCTCGAGAAAAGAGAAGCCATGGTAGGTT-3′
Forward (−400)
(SEQ ID NO: 19)
5′-GCGCGCTCGAGCACATATGCACGAGAGAGACAG

The same reverse primer is used for all PCR reactions. The numbers in parenthesis represent the location of the 5′ end of the primer in the promoter sequence.

### Example 5

### Characterization of Binding Sites in Agg-1 Promoter Gene by EMSA

Wild type and mutant oligos representing NF-kB, STAT3 and AP-1 transcription factor binding sites for EMSA are synthesized and labeled as follows:

Double-stranded oligos representing the NF-kB, STAT3 and AP-1 sites are synthesized by Sigma Genosys, Woodlands, Tex.

### NF-kB (−1799) Wild Type Oligo

Forward

| | |
|---|---|
| 5′-CCTTCCTGGGGATTTCCTGGGG-3′ | (SEQ ID NO: 20) |
| Reverse | |
| 5′-CCCCAGGAAATCCCCAGGAAGG-3′ | (SEQ ID NO: 21) |

### NF-kB (−1799) Mutant Oligo

Forward

| | |
|---|---|
| 5′-CCTTCCTGGaGATTTCCTGGGG-3′ | (Seq ID No. 22) |
| Reverse | |
| 5′-CCCCAGGAAATCtCCAGGAAGG-3′ | (Seq ID No. 23) |

### AP-1 (−2195) Wild Type Oligo

| Forward | |
|---|---|
| 5′-CATTGCTTAGTCACCCCCTT-3′ | (SEQ ID NO: 24) |
| Reverse | |
| 5′-AAGGGGGTGACTAAGCAATG-3′ | (SEQ ID NO: 25) |

### AP-1 (−2195) Mutant Oligo

| Forward | |
|---|---|
| 5′-CATTGCTTgGgCACCCCCTT-3′ | (SEQ ID NO: 26) |
| Reverse | |
| 5′-AAGGGGGTGcCcAAGCAATG-3′ | (SEQ ID NO: 27) |

### STAT3 (−1841) Wild Type Oligo

Forward

| | |
|---|---|
| 5′-GGTCCACTTCTGGGAAAGGAAAGAGAC-3′ | (SEQ ID NO: 28) |
| Reverse | |
| 5′-GTCTCTTTCCTTTCCCAGAAGTGGACC-3′ | (SEQ ID NO: 29) |

### STAT3 (−1841) Mutant Oligo

Forward

| | |
|---|---|
| 5′-GGTCCACaTaTGGGAAAGGAAAGAGAC-3′ | (SEQ ID NO: 30) |
| Reverse | |
| 5′-GTCTCTTTCCTTTCCCAtAtGTGGACC-3′ | (SEQ ID NO: 31) |

Nuclear extracts are prepared as follows: HAC, HeLa or SW 1.353 cells are grown to confluency in 150 mM dishes in DMEM high glucose medium supplemented with 10% fetal bovine serum, penicillin, streptomycin and fungizone. The cells are serum starved for an hour and induced with IL-1 (2 ng/mL) alone, OSM (40 ng/mL) alone, IL-1 and OSM and/or left untreated for three hours. Cells are re-suspended in a hypotonic buffer containing 10% glycerol, 20 mM HEPES (pH 7.9), 10 mM KCl, 1 mM EDTA, 1 mM DTT, 0.2% NP40, 0.1 mM sodium vanadate, 20 mM sodium fluoride and a protease cocktail (Sigma, St, Louis, Mo.) and incubated on ice for 10 minutes. The nuclear fraction is recovered as a pellet by centrifugation at 13,000 rpm in an eppendorf centrifuge. The pellet is re-suspended in a hypertonic buffer containing 20% glycerol, 20 mM HEPES (pH 7.9), 10 mM KCl, 420 mM NaCl, 1 mM EDTA and 1 mM DTT and incubated on ice for 30 Minutes and once again centrifuged. The supernatant is collected as the nuclear extract.

### Electrophoretic Mobility Shift Assays

The EMSA is performed as follows: NF-Kb, STAT3 and AP-1 double-stranded oligos are generated by annealing appropriate single-stranded oligo pairs. The oligos are denatured by heating in annealing buffer (10 mM Tris-HCl pH 7.5 containing 1 mM EDTA, 30 mM NaCl) at 65° C. for 5 minutes followed by slow cooling to room temperature. The double-stranded oligos are phosphorylated using [γ-³²]-ATP and T4 polynucleotide kinase using the procedures and reagents provided in the gel shift assay systems kit (Promega, Madison, Wis.). Free nucleotides are removed using Sephadex G-25 spin columns (Pharmacia Biotech, Piscataway, N.J.).

### NF-kB and STAT3 EMSA

5-8 μg of nuclear extracts are used in each reaction. Nuclear extracts are pre-incubated with cold competitors for 5 minutes at 4° C. in binding buffer (20 mM HEPES pH 7.9, 40 mM KCl, 1.0 mM MgCl₂, 0.1 mM EGTA, 0.5 mM DTT and 4% Ficoll). The reaction is initiated by the addition of 1 μL of ³²P-labeled probes (2×10⁴ cpm) in the presence of 1 μg of poly (dI-dC). Reactions are incubated at room temperature for 30 minutes. Samples are eletrophoresed on 4% polyacrylamide, 0.25×TBE gels at 120 volts. Gels are dried and subjected to autoradiography at −70° C. using Kodak X-OMAT AR X-Ray film. For antibody supershift assays, 2 μg of NFkB-p50 and 2 μg of STAT 1, 3 or 5 antibodies (Santa Cruz Biotech, Santa Cruz, Calif.) are pre-incubated with 5-8 μg of nuclear extracts for 10 minutes on ice before the addition of radiolabelled oligonucleotide probe. The products are analyzed by PAGE as described above.

### AP-1 EMSA

The assay is performed as described above for NF-kB except that standard binding buffer provided in the Promega gel shift assay systems kit is used (10 mM Tris-HCl pH 7.5, 1 mM MgCl₂, 0.5 mM EDTA, 0.5 mMDTT, 60 mM NaCl, 50 μg/mL poly (dI-dC) and 4% glycerol).

To identify Fos and Jun components of the AP-1 complex antibody supershifts are carried out. About 2 μg of nuclear extracts are pre-incubated with 2 μg of antibodies (Santa Cruz Biotech) against c-Jun, JunB, JunD, c-Fos, FosB, Fm 1,2 and Creb 1,2/ATF-1,2,3, for 30 minutes at room temperature before the addition of the raft-labeled oligonucleotide probe. The products are analyzed by polyacrylamide gel electrophoresis as described above.

### Example 5

### Site Directed Mutagenesis

Single- or double-mutations of AP-1 (−2195), STAT 3 (−1841) and NF-kB (−1800) sites in 2.4 kb construct are generated using the Quick Change site, Directed Mutagenesis Kit (Stratagene, La Jolla, Calif.) as per manufacturers instruction. The following primers are used in the PCR.

Forward AP-1
(SEQ ID NO: 32)
5′-CTTGTCTTTCATTGCTTgGgCACCCCCTTTGTCCT-3′
Reverse AP-1
(SEQ ID NO: 33)
5′-GAGGACAAAGGGGGTGcCcAAGCAATGAAAGACAAAG-3′
Forward STAT3
(SEQ ID NO: 34)
5′-CAAGAGGAGGTGGTCCACaTaTGGGAAAGGAAAGAGAC-3′
Reverse STAT3
(SEQ ID NO: 35)
5′-GTCTCTTTCCTTTCCCAtAtGTGGACCACCTCCTCTTG-3′
Forward NF-kB
(SEQ ID NO: 36)
5′-CACTCTCCTTCCTGGaGATTTCCTGGGGAAAC-3′
Reverse NF-kB
(SEQ ID NO: 37)
5′-GTTTCCCCAGGAAATCtCCAGGAAGGAGAGTG-3′

Site specific mutations is confirmed by DNA sequencing. The 2.4 kb inserts are cut out with Xho I and Hind III and redoned into pGL3 basic Luciferase vector digested with Xho I and Hind III.

### Example 6

### Transfection of Agg-1 Promoter Constructs into Cell Lines

SW 1353 (human chondrosarcoma cells), HeLa, HEK 293 and NIH 3T3 cells are transiently transfected using Lipofectamine 2000 reagent (Invitrogen, Carlsbad, Calif.) according to procedures described by the vendor. Briefly, cells are seeded at 2×10⁵/well in 6-well plates and grown overnight in 3 mL DMEM high glucose medium supplemented with 10% fetal bovine serum, penicillin, streptomycin and fungizone (Invitrogen). SV-40 transformed chondrocyte cells—C28A2 (see Koshy et al. (2002), supra) cells are transfected by CaPO₄ method using MBS Mammalian Transfection Kit (Stratagene, La Jolla, Calif.) according to procedure described in their manual. Total of 4 μg of plasmid DNA/well (3.5 μg reporter plasmid and 0.5 μg CMV β-Gal) is used for transfections. Cells are changed to fresh media 5 hours post-transfection and incubated for about 40 hours for expression of transfected reporters.

### IL-1 and OSM Treatment of Transfected Cells

The cells are serum starved for 1-2 hours, 40 hours post-transfection and induced with IL-1 2 ng/mL (Peprotech, Rockyhill, N.J.) and OSM 40 ng/mL (R&D systems, Minneapolis, Minn.) for 4 hours.

### Example 7

### Luciferase Assay

The cells are washed with phosphate buffered saline (PBS) and 400 μL of Glow lysis buffer (Promega, Madison, Wis.) are added to the cells. The cells are lysed by repeated re-suspension in the buffer and transferred to eppendorf tubes. One hundred (100) μL of the cell lysates are added to 96-well plates (Falcon, white opaque bottom) followed by the addition of 100 μL of bright glow luciferase assay reagent (Promega, Madison, Wis.). The plates are read in a MLX microtiter plate luminometer (Dynex Technologies, Chantilly, Va.).

### Example 8

### β-Galatosidase Assay

Fifty (50) μL of cell lysates are pipetted into 96-well plates (Costar, clear bottom, black) and 50 μL of 2×n-Gal assay buffer (Promega, Madison, Wis.) are added and incubated at 37° C. until yellow color developed in all wells. The reaction is stopped by the addition of 150 μL 1 M tris-HCl, pH 7.5. The absorbance of the samples is measured at 420 nm in a plate reader (Molecular Devices, Sunny Valley, Calif.).

The inducibility by cytokinesis most effective in SW1353 and HeLa Growth factors, such as PDGF are poor inducers of the promoter in these cells.

Analysis of deletion variants suggests that the region between −0.4 kb and −0.8 kb is important for minimal promoter activity and the segment between −1.2 kb and −2.4 kb is important for inducibility by IL-1 and OSM. Site directed mutational analysis reveals that STATS (−1841) and NFkB (−1799) sites are central for inducibility by IL-1 and OSM whereas mutation of the AP-1 (−2195) site did not alter responsiveness to IL-1 and OSM.

What is claimed is:

1. An isolated polynucleotide comprising a nucleic acid sequence selected from the group consisting of SEQ ID NOs: 1-6 or degenerate variants thereof.

2. An isolated nucleic acid molecule comprising any one of the following:
(a) a nucleic acid sequence at least 90% identical to a nucleic acid sequence selected the group consisting of SEQ ID NOs: 1-6;
(b) a complement of the nucleic acid sequence of (a) above; or
(c) a fragment of the nucleic acid sequence of (a) or (b) above wherein the fragment comprises at least 20 nucleotides.

3. An isolated nucleic acid molecule comprising a nucleic acid sequence that hybridizes under stringent conditions to a hybridization probe, the nucleic acid sequence of which is selected from the group consisting of SEQ ID NOs: 1-6 or a complement thereof.

4. An isolated polynucleotide that encodes an Aqq-1 promoter gene that drives expression, said Agg-1 promoter gene comprising a nucleotide sequence selected from the group consisting of:
(a) a nucleotide sequence selected from the group consisting of SEQ ID NO. 1, SEQ ID NO. 2, SEQ ID NO. 3, SEQ ID NO. 4, SEQ ID NO. 5, and SEQ ID NO. 6, or a fragment or variant thereof;
(b) a nucleotide sequence having at least 80% identity to a nucleotide sequence selected from the group consisting of SEQ ID NO. 1, SEQ ID NO. 2, SEQ ID NO 3, SEQ ID NO. 4, SEQ ID NO. 5, and SEQ ID NO. 6;
(c) a nucleotide sequence having at least 90% identity to a nucleotide sequence selected from the group consisting of SEQ ID NO. 1, SEQ ID NO. 2, SEQ ID NO. 3, SEQ ID NO. 4, SEQ ID NO. 5, and SEQ ID NO. 6;
(d) a nucleotide sequence having at least 95% identity to the nucleotide sequence selected from the group consisting of SEQ ID NO. 1, SEQ ID NO. 2, SEQ ID NO. 3, SEQ ID NO. 4, SEQ ID NO. 5, and SEQ ID NO. 6;
(e) a nucleotide sequence selected from the group consisting of SEQ ID NO. 1, SEQ ID NO. 2, SEQ ID NO. 3, SEQ ID NO. 4, SEQ ID NO. 5, and SEQ ID NO. 6;
(f) a nucleotide sequence having at least 10 contiguous nucleotides of a sequence selected from the group consisting of SEQ ID NO. 1, SEQ ID NO. 2, SEQ ID NO. 3, SEQ ID NO. 4, SEQ ID NO. 5, and SEQ ID NO. 6;
(g) a nucleotide sequence having at least 20 contiguous nucleotides of the sequence selected from the group consisting of SEQ ID NO. 1, SEQ ID NO. 2, SEQ ID NO. 3, SEQ ID NO. 4, SEQ. ID NO. 5, and SEQ ID NO. 6; and
(h) a nucleotide sequence that hybridizes to the nucleotide sequence set forth in (a)-(g) above under stringent conditions.

5. (canceled)

6. A vector comprising the Agg-1 promoter gene as in claim 4.

7. A host cell transfected or transformed with the vector of claim 6.

8-24. (canceled)

25.-37. (canceled)

38. A method for producing an expression product encoded by a nucleic acid molecule comprising culturing a host cell, which has been transfected or transformed with an expression vector comprising an Agg-1 promoter gene that drives expression of the nucleic acid molecule, under conditions suitable for expression of the nucleic acid molecule and recovering an expression product produced, said Agg-1 promoter gene comprising a nucleotide sequence selected from the group consisting of
(a) a nucleotide sequence selected from the group consisting of SEQ ID NO. 1, SEQ ID NO. 2, SEQ ID NO. 3, SEQ ID NO. 4, SEQ ID NO. 5, and SEQ ID NO. 6, or a fragment or variant thereof;
(b) a nucleotide sequence having at least 80% identity to a nucleotide sequence selected from the group consisting of SEQ ID NO. 1, SEQ ID NO. 2, SEQ ID NO. 3, SEQ ID NO. 4, SEQ ID NO. 5, and SEQ ID NO. 6;
(c) a nucleotide sequence having at least 90% identity to a nucleotide sequence selected from the group consisting of SEQ ID NO. 1, SEQ ID NO. 2, SEQ ID NO. 3, SEQ ID NO. 4, SEQ ID NO. 5, and SEQ ID NO. 6;
(d) a nucleotide sequence having at least 95% identity to the nucleotide sequence selected from the group consisting of SEQ ID NO. 1, SEQ ID NO. 2, SEQ ID NO. 3, SEQ ID NO. 4, SEQ ID NO. 5, and SEQ ID NO. 6;
(e) a nucleotide sequence having at least 10 contiguous nucleotides of a sequence selected from the group consisting of SEQ ID NO. 1, SEQ ID NO. 2, SEQ ID NO. 3, SEQ ID NO. 4, SEQ ID NO. 5, and SEQ ID NO. 6;
(f) a nucleotide sequence having at least 20 contiguous nucleotides of the sequence selected from the group consisting of SEQ ID NO. 1, SEQ ID NO. 2, SEQ ID NO. 3, SEQ ID NO. 4, SEQ ID NO. 5, and SEQ ID NO. 6; and
(g) a nucleotide sequence that hybridizes to the nucleotide sequence set forth in (a) to (f) above under stringent conditions.

39. The method of claim 38, wherein said expression is constitutive.

40. The method of claim 38, wherein said expression is inducible.

41. The method of claim 38, wherein the host cell is a prokaryotic or a eukaryotic host cell.

42. The method of claim 41, wherein the host cell is a nonchondrocytic cell.

43. The method of claim 38, wherein the expression product is a nucleic acid molecule, a peptide, a polypeptide or a protein.

44. The method of claim 43, wherein the expression product is a human protein or a fragment thereof.
